(19) United States
(12) Patent Application Publication
(10) Pub. No.: US 2022/0202044 A1
(43) Pub. Date: Jun. 30, 2022

(54) FIBROUS MAIZE-BASED ANIMAL FEED WITH GH30 GLUCURONOXYLAN HYDROLASE

(71) Applicant: Novozymes A/S, Bagsvaerd (DK)

(72) Inventors: Eduardo Antonio Della Pia, Lyngby (DK); Jonas Ravn, Bagsvaerd (DK); Ninfa Rangel Pedersen, Soeborg (DK)

(73) Assignee: Novozymes A/S, Bagsvaerd (DK)

(21) Appl. No.: 17/436,726

(22) PCT Filed: Mar. 5, 2020
(86) PCT No.: PCT/EP2020/055830

§ 371 Date: Sep. 7, 2021

(30) Foreign Application Priority Data
Mar. 11, 2019 (EP) 19161908.9

(51) Int. Cl. A23K 10/14; A23K 10/12; A23K 20/189; A23K 50/75; A23K 50/30; A61K 36/899; A61K 9/00; A61K 38/47; A61P 1/14; A61K 35/742

(52) U.S. Cl. CPC A23K 10/14; A23K 10/12; A23K 20/189; A23K 50/75; A61K 35/742; A61K 36/899; A61K 9/0056; A61K 38/47; A61P 1/14; A23K 50/30

(57) ABSTRACT

Improvement of the intestinal health of a monogastric animal by increasing the levels of cecal butyrate levels in situ in said animal by administering a GH30 glucuronoxylan hydrolase-enriched maize-based animal feed is described. Improving the feed conversion ratio of said animal feed is furthermore observed.

### REFERENCE TO SEQUENCE LISTING This application contains a Sequence Listing in computer readable form. The computer readable form is incorporated herein by reference.

### FIELD OF THE INVENTION

The present invention relates to improving the intestinal health of an animal comprising the administration of a GH30 glucuronoxylan hydrolase-enriched maize-based animal feed and improving the feed conversion ratio of said animal feed by means of the administration of a GH30 glucuronoxylan hydrolase.

### BACKGROUND OF THE INVENTION

Maintaining a healthy gut is important in broiler chicken production, and together with environmental conditions, the diet is the key contributing factor affecting the microbiota composition. Broiler diets consist mainly of cereals and vegetable protein sources containing various amounts of fibre which may be highly indigestible depending on the plant species.

Plant fibres essentially are composed of polysaccharides other than starch, also termed non-starch polysaccharides (NSP). The main NSP in maize are glucurono-arabinoxylans (GAX) which have a highly recalcitrant, insoluble and heterogenous structure). NSP degrading feed enzymes hydrolyse and solubilise insoluble xylan, generating prebiotic oligomers such as arabinoxylan oligosaccharides (AXOS). These soluble low molecular weight AXOS pass through the gastrointestinal tract (GIT) and pass into the large intestine for fermentation. Prebiotic AXOS may be produced directly in situ by enzymes, which may increase their fermentability in the hindgut. Some of the end products of bacterial fermentation are known to improve gut health. Especially increased formation of butyrate may indicate a better health status of the gut, since butyrate is a well-known gut health promoting molecule with anti-inflammatory properties.

For many of the NSP degrading enzymes, the exact mode of action in the GIT is not fully understood. Therefore, the inventors investigated the effect of supplementing exogenous glucuronoxylan hydrolase from GH30 family targeting the maize GAX to maize fibre fermented with a microbial inoculum from broiler ceca.

Intestinal dysbiosis, defined as an imbalance between harmful and beneficial bacteria in the gut have increased in broiler production since the ban of feed antimicrobials. This had led to an increase in sporadic cases of Clostridium perfringens-associated necrotic enteritis and other enteric diseases resulting in decreased growth performance in broiler chickens.

### SUMMARY OF THE INVENTION

According to the invention, Glucuronoxylan Hydrolase from glycoside hydrolase family 30 (GH30) promotes microbial diversity and butyrate production in cecal broiler fermentations of maize fibre.

The xylanases currently on the market are effective on wheat-based diets and not corn-based diets. However, corn is the preferred cereal source in monogastric diets. The ability of glucuronoxylan hydrolase from GH30 family to solubilize corn arabinoxylan to produce oligomers that can be used to produce short chain fatty acid by gut microbiota is an important aspect of the present invention and the increase in growth performance and intestinal gut parameters from corn-based diets enhanced with glucuronoxylan hydrolase from GH30 family is further aspect of the present invention.

An objective of the present disclosure is to demonstrate the broiler growth performance and gut health benefits from supplementing a maize/soy/DDGS diet with an exogenous monocomponent glucuronoxylan hydrolase targeting maize GAX. A further objective is to demonstrate the effect of the generated maize AXOS on butyrate production and broiler microbiota composition.

One aspect of the invention is directed to a method of improving the feed conversion ratio of an animal feed comprising maize and adding GH30 glucuronoxylan hydrolase to said animal feed. A related aspect is directed to a method of improving the feed conversion ratio of monogastric animals comprising the use of GH30 glucuronoxylan hydrolase in a maize-based animal feed. A further related aspect is directed to a use of GH30 glucuronoxylan hydrolase to prepare an enzyme-enriched animal feed, wherein said animal feed is a maize-based animal feed.

A further aspect is directed to an enzyme-enriched animal feed comprising GH30 glucuronoxylan hydrolase and maize wherein the feed comprises maize in an amount of 100 to 1000 g/kg feed and GH30 glucuronoxylan hydrolase in an amount of 2 to 100 ppm per kg of feed.

An alternative aspect of the invention is directed to a method of generating a prebiotic in-situ in a maize-based animal feed comprising the use of a GH30 glucuronoxylan hydrolase added to said feed. Similarly, a further aspect is directed to a method of improving the intestinal health of a monogastric animal by in-situ production of arabinoxylan oligosaccharides and polysaccharides. Alternatively defined, the aspect of the invention may be expressed as a method for the in-situ production of prebiotics in monogastric animals comprising the administration of an enzyme-enriched maize-based animal feed to said animal wherein said animal feed comprises the enzyme GH30 glucuronoxylan hydrolase.

The invention is further directed to a method of decreasing the insoluble maize fraction in a maize-based animal feed comprising the addition of a GH30 glucuronoxylan hydrolase.

An interesting aspect of the invention is a method of improving the intestinal health of a monogastric animal comprising the administration of an enzyme-enriched maize-based animal feed to said animal wherein said animal feed comprises the enzyme GH30 glucuronoxylan hydrolase.

A further aspect of the invention is directed to a method for improving intestinal health in a monogastic animal, said method comprising increasing the levels of cecal butyrate levels in situ in said animal said method comprising the administration of an enzyme-enriched maize-based animal feed to said animal wherein said animal feed comprises the enzyme GH30 glucuronoxylan hydrolase. Similarly expressed, the invention is directed to a method for improving intestinal health in a monogastic animal said method comprising altering the microbiota composition in said animal by administration of an enzyme-enriched maize-based animal feed to said animal wherein said animal feed comprises the enzyme GH30 glucuronoxylan hydrolase. A related aspect is directed to a method of improving the intestinal health of a monogastric animal comprising the administration of an enzyme-enriched maize-based animal feed to said animal wherein said animal feed comprises the enzyme GH30 glucuronoxylan hydrolase.

A related aspect of the invention is directed to a method of causing a butyrogenic effect in a monogastric animal comprising the administration of an enzyme-enriched maize-based animal feed to said animal wherein said animal feed comprises the enzyme GH30 glucuronoxylan hydrolase.

### BRIEF DESCRIPTION OF DRAWINGS

FIG. 1 shows the size exclusion chromatography of enzymatic digest of maize fibre using the Superdex 75 column. Pool I was composed of fraction 22-30 (10-30 kD), Pool II was composed of fraction 31-39 (4-10 kD), pool III was composed of fraction 40-53 (1-4 kD), pool IV was composed of fraction 55-59 (100-500 Da) and pool V was composed of fraction 60-70 (<100 D). The black line indicates RI-index and the grey line indicates UV-index.

FIG. 2 shows the boxplot of alpha (Shannon) diversity for the control samples and the samples treated with GH30.

FIG. 3 shows the PCA visualisation of beta diversity for the 4 treatment types. Unifrac distances were used as distances.

FIG. 4 shows the heatmap visualization of hierarchical clustering of the top 10 most abundant species. Colour coding from blue to red indicate log transformed relative abundances.

FIG. 5 shows the abundance of the Bacteroides species OTU3 (xylanisolvens) and OTU5 (dorei/vulgatus).

FIG. 6 shows the heatmap visualization of hierarchical clustering of the top 10 most abundant genera. Colour coding from blue to red indicate log transformed relative abundances.

FIG. 7 shows the relative abundance of genus Bifidobacterium and Faecalibacterium.

FIG. 8 shows the PCA visualisation of beta diversity for the 4 fractions. Unifrac distances were used as distances.

FIG. 9 shows the heatmap visualization of hierarchical clustering of the top 20 most abundant species. Color coding from blue to red indicate log transformed relative abundances

FIG. 10 shows the abundance of the Bacteroides species OTU5 (family Ruminococcaceae), OTU15 (Family Lachnospiraceae), OTU10 (genus Faecalibacterium) and OTU3 (genus Bacteroides).

FIG. 11 shows the ratio between butyryl-CoA:acetate-CoA transferase gene and total bacteria copies in cecal content of 29-day old chickens supplemented with GH30 of SEQ ID NO 1 (GXH) or not (Control). A Tukey-Kramer HSD test was done to compare all means of pairs between groups receiving non-supplemented and enzyme-supplemented diets. Each dot represents one individual bird. P values of less than 0.05 (*) were considered significant.

### SEQUENCES

SEQ ID NO 1 is a mature polypeptide Bacillus subtilis GH30 xylanase, expressed in the Bacillus licheniformis host cells for production

MIPRIKKTICVLLVCFTMLSVMLGPGATEVLAASDVTVNVSAEKQVIRG
FGGMNHPAWAGDLTAAQRETAFGNGQNQLGFSILRIHVDENRNNVVYKE
VETAKSAVKHGAIVFASPWNPPSDMVETFNRNGDTSAKRLKYNKYAAYA
QHLNDFVTFMKNNGVNLYAISVQNEPDYAHEVVTWVVTPQEILRFMREN
AGSINARVIAPESFQYLKNLSDPILNDPQALANMDILGTHLYGTQVSQF
PYPLFKQKGAGKDLWMTEVYYPNSDTNSADRWPEALDVSQHIHNAMVEG
DFQAYVVWVYIRRSYGPMKEDGTISKRGYNMAHFSKFVRPGYVRIDATK
NPNANVYVSAYKGDNKVVIVAINKSNTGVNQNFVLQNGSASNVSRWITS
SSSNLQPGTNLTVSGNHFWAHLPAQSVTTFVVNR

SEQ ID NO: 2 is a Bacillus subtilis GH30 xylanase and variant of SEQ ID NO 1 with the following mutations: H24W/V74L/H76L/I155M/V208L, counting after the signal peptide comorisina the seauence:

AASDVTVNVSAEKQVIRGFGGMNWPAWAGDLTAAQRETAFGNGQNQLGF
SILRIHVDENRNNVVYKEVETAKSALKLGAIVFASPWNPPSDMVETFNR
NGDTSAKRLKYNKYAAYAQHLNDFVTFMKNNGVNLYAISVQNEPDYAHE
WTWWTPQEMLRFMRENAGSINARVIAPESFQYLKNLSDPILNDPQALAN
MDILGTHLYGTQLSQFPYPLFKQKGAGKDLWMTEVYYPNSDTNSADRWP
EALDVSQHIHNAMVEGDFQAYVWWYIRRSYGPMKEDGTISKRGYNMAHF
SKFVRPGYVRIDATKNPNANVYVSAYKGDNKVVIVAINKSNTGVNQNFV
LQNGSASNVSRWITSSSSNLQPGTNLTVSGNHFWAHLPAQSVTTFVVNR

SEQ ID NO: 3 is a Bacillus subtilis GH30 xylanase comprising the sequence

AASDATVRLSAEKQVIRGFGGMNHPAWIGDLTAAQRETAFGNGQNQLGF
SILRIHVDENRNNVVYREVETAKSAIKHGAIVFASPWNPPSDMVETFNR
NGDTSAKRLRYDKYAAYAKHLNDFVTFMKNNGVNLYAISVQNEPDYAHD
VVTVWVTPQEILRFMKENAGSINARVIAPESFQYLKNISDPIVNDPKAL
ANMDILGAHLYGTQLNNFAYPLFKQKGAGKDLWMTEVYYPNSDNHSADR
WPEALDVSHHIHNSMVEGDFQAYVVWVYIRRSYGPMKEDGTISKRGYNM
AHFSKFVRPGYVRVDATKSPASNVYVSAYKGDNKVVIVAINKNNSGVNQ
NFVLQNGSVSQVSRWITSSSSNLQPGTNLNVTDNHFWAHLPAQSVTTFV
ANLR

SEQ ID NO 4 comprises the sequence:

ANTDYWQNVVTDGGGTVNAVNGSGGNYSVNWSNTGNFVVGKGVVTTGSP
FRTINYNAGVWAPNGNAYLTLYGVVTRSPLIEYYVVDSWGTYRPTGTYK
GTVYSDGGTYDVYTTTRYDAPSIDGDKTTFTQYWSVRQSKRPTGSNATI
TFSNHVNAWKRYGMNLGSNWSYQVLATEGYRSSGSSNVTVW

SEQ ID NO 5 comprises the sequence:

ASTDYWQNVVTDGGGIVNAVNGSGGNYSVNWSNTGNFVVGKGVVTTGSP
FRTINYNAGVWAPNGNGYLTLYGVVTRSPLIEYYVVDSWGTYRPTGTYK
GTVKSDGGTYDIYTTTRYNAPSIDGDRTTFTQYWSVRQSKRPTGSNATI
TFSNHVNAWKSHGMNLGSNWAYQVMATEGYQSSGSSNVTVW

SEQ ID NO: 6 is the amino acid sequence of the mature GH30_8 xylanase from Clostridium acetobutylicum:

SEQ ID NO: 7 is the amino acid sequence of the mature GH30_8 xylanase from Pseudoalteromonas tetraodonis:

SEQ ID NO: 8 is the amino acid sequence of the mature GH30_8 xylanase from Paenibacillus sp-19179:

SEQ ID NO: 9 is the amino acid sequence of the mature GH30_8 xylanase Pectobacterium carotovorum subsp. Carotovorum:

SEQ ID NO: 10 is the amino acid sequence of the mature GH30_8 xylanase Ruminococcus sp. CAG:330

SEQ ID NO: 11 comprises the amino acid sequence of the mature GH30_8 xylanase Streptomyces sp-62627: Arg Leu Pro Ala Gln Ser Val Thr Thr Leu Val Thr Gly

SEQ ID NO: 12 is the amino acid sequence of the mature GH30_8 xylanase Clostridium saccharobutylicum:

SEQ ID NO: 13 is the amino acid sequence of the mature GH30_8 xylanase Paenibacillus panacisoli.

SEQ ID NO: 14 is the amino acid sequence of the mature GH30_8 xylanase Human Stool metagenome

SEQ ID NO: 15 is the amino acid sequence of the mature GH30_8 xylanase Vibrio rhizosphaerae:

SEQ ID NO: 16 is the amino acid sequence of a mature GH30 xylanase from Bacillus subtilis.

SEQ ID NO: 17 is the amino acid sequence of a mature GH30 xylanase from Bacillus amyloliquefaciens.

SEQ ID NO: 18 is the amino acid sequence of a mature GH30 xylanase from Bacillus licheniformis.

SEQ ID NO: 19 is the amino acid sequence of a mature GH30 xylanase from Bacillus subtilis.

SEQ ID NO:20 is the amino acid sequence of a mature GH30 xylanase from Paenibacillus pabuli.

SEQ ID NO: 21 is the amino acid sequence of a mature GH30 xylanase from Bacillus amyloliquefaciens HB-26.

### DETAILED DESCRIPTION OF THE INVENTION

For the first time it has been shown that a GH30 xylanase targeting insoluble and highly substituted maize glucuronoarabinoxylan can improve intestinal functionality in broiler chickens fed with a fibrous maize-soy bean meal diet. Since maize has highly branched arabinoxylan, it is not easy for xylanases to solubilize the same. It was also not expected that gut bacteria could further breakdown the highly branched oligomers and convert them to volatile fatty acids which are known to enhance GI tract functionality.

Animal: The term “animal” refers to all animals except humans. Examples of animals are non-ruminants, and ruminants. Ruminant animals include, for example, animals such as sheep, goats, cattle, e.g. beef cattle, cows, and young calves, deer, yank, camel, llama and kangaroo. Non-ruminant animals include mono-gastric animals, e.g. pigs or swine (including, but not limited to, piglets, growing pigs, and sows); poultry such as turkeys, ducks and chicken (including but not limited to broiler chicks, layers); horses (including but not limited to hotbloods, coldbloods and warm bloods), young calves; fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns).

Animal feed: The term “animal feed” refers to any compound, preparation, or mixture suitable for, or intended for intake by an animal. Animal feed for a mono-gastric animal typically comprises concentrates as well as vitamins, minerals, enzymes, direct fed microbial, amino acids and/or other feed ingredients (such as in a premix) whereas animal feed for ruminants generally comprises forage (including roughage and silage) and may further comprise concentrates as well as vitamins, minerals, enzymes direct fed microbial, amino acid and/or other feed ingredients (such as in a premix).

Body Weight Gain: The term “body weight gain” means an increase in live weight of an animal during a given period of time e.g. the increase in weight from day 1 to day 21.

Composition: The term “composition” refers to a composition comprising a carrier and at least one enzyme of the present invention. The compositions described herein may be mixed with an animal feed and referred to as a “mash feed.”

Effective amount/concentration/dosage: The terms “effective amount”, “effective concentration”, or “effective dosage” are defined as the amount, concentration, or dosage of the enzyme(s) sufficient to improve the digestion or yield of an animal. The actual effective dosage in absolute numbers depends on factors including: the state of health of the animal in question, other ingredients present. The “effective amount”, “effective concentration”, or “effective dosage” of the enzyme(s) may be determined by routine assays known to those skilled in the art.

Feed Conversion Ratio: The term “feed conversion ratio” defines the amount of feed fed to an animal to increase the weight of the animal by a specified amount. An improved feed conversion ratio means a lower feed conversion ratio. By “lower feed conversion ratio” or “improved feed conversion ratio” it is meant that the use of a feed additive composition in feed results in a lower amount of feed being required to be fed to an animal to increase the weight of the animal by a specified amount compared to the amount of feed required to increase the weight of the animal by the same amount when the feed does not comprise said feed additive composition.

Feed efficiency: The term “feed efficiency” means the amount of weight gain per unit of feed when the animal is fed ad-libitum or a specified amount of food during a period of time. By “increased feed efficiency” it is meant that the use of a feed additive composition according the present invention in feed results in an increased weight gain per unit of feed intake compared with an animal fed without said feed additive composition being present.

Nutrient Digestibility: The term “nutrient digestibility” means the fraction of a nutrient that disappears from the gastro-intestinal tract or a specified segment of the gastro-intestinal tract, e.g. the small intestine. Nutrient digestibility may be measured as the difference between what is administered to the subject and what comes out in the faeces of the subject, or between what is administered to the subject and what remains in the digesta on a specified segment of the gastro intestinal tract, e.g. the ileum.

Nutrient digestibility as used herein may be measured by the difference between the intake of a nutrient and the excreted nutrient by means of the total collection of excreta during a period of time; or with the use of an inert marker that is not absorbed by the animal, and allows the researcher calculating the amount of nutrient that disappeared in the entire gastro-intestinal tract or a segment of the gastro-intestinal tract. Such an inert marker may be titanium dioxide, chromic oxide or acid insoluble ash. Digestibility may be expressed as a percentage of the nutrient in the feed, or as mass units of digestible nutrient per mass units of nutrient in the feed. Nutrient digestibility as used herein encompasses starch digestibility, fat digestibility, protein digestibility, and amino acid digestibility.

Energy digestibility as used herein means the gross energy of the feed consumed minus the gross energy of the faeces or the gross energy of the feed consumed minus the gross energy of the remaining digesta on a specified segment of the gastro-intestinal tract of the animal, e.g. the ileum. Metabolizable energy as used herein refers to apparent metabolizable energy and means the gross energy of the feed consumed minus the gross energy contained in the faeces, urine, and gaseous products of digestion. Energy digestibility and metabolizable energy may be measured as the difference between the intake of gross energy and the gross energy excreted in the faeces or the digesta present in specified segment of the gastro-intestinal tract using the same methods to measure the digestibility of nutrients, with appropriate corrections for nitrogen excretion to calculate metabolizable energy of feed.

Pellet: The terms “pellet” and/or “pelleting” refer to solid rounded, spherical and/or cylindrical tablets or pellets and the processes for forming such solid shapes, particularly feed pellets and solid extruded animal feed. As used herein, the terms “extrusion” or “extruding” are terms well known in the art and refer to a process of forcing a composition, as described herein, through an orifice under pressure.

Poultry: The term “poultry” means domesticated birds kept by humans for the eggs they produce and/or their meat and/or their feathers. Poultry includes broilers and layers. Poultry include members of the superorder Galloanserae (fowl), especially the order Galliformes (which includes chickens, Guineafowls, quails and turkeys) and the family Anatidae, in order Anseriformes, commonly known as “waterfowl” and including domestic ducks and domestic geese. Poultry also includes other birds that are killed for their meat, such as the young of pigeons. Examples of poultry include chickens (including layers, broilers and chicks), ducks, geese, pigeons, turkeys and quail.

Roughage: The term “roughage” means dry plant material with high levels of fiber, such as fiber, bran, husks from seeds and grains and crop residues (such as stover, copra, straw, chaff, sugar beet waste).

Ruminant: The term “ruminant” means a mammal that digests plant-based food by initially fermenting/degrading it within the animal's first compartment of the stomach, principally through bacterial actions, then regurgitating the semi-digested mass, now known as cud, and chewing it again. The process of re-chewing the cud to further break down plant matter and stimulate digestion is called “ruminating”. Examples of ruminants are cattle, cow, beef cattle, young calf, goat, sheep, lamb, deer, yank, camel and llama.

SCFA: The term “SOFA” is an abbreviation for Short-Chain Fatty Acid. SCFAs are fatty acids with fewer than six carbon atoms and are derived from intestinal microbial fermentation of indigestible foods, SCFAs are the main energy source of colonocytes, making them crucial to gastrointestinal health. In the present invention, the SOFA can be selected from the group consisting of a methanoate, an acetate, a propionate, a butyrate, an isobutyrate, a valerate and an isovalerate.

Swine: The term “swine” or “pigs” means domesticated pigs kept by humans for food, such as their meat. Swine includes members of the genus Sus, such as Sus scrofa domesticus or Sus domesticus and include piglets, growing pigs, and sows.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), e.g., version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), e.g., version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides x 100)/(Length of Alignment−Total Number of Gaps in Alignment)

Glucuronoxylan Hydrolases

An aspect of the invention is directed to a method of improving the feed conversion ratio of an animal feed comprising maize comprising adding GH30 glucuronoxylan hydrolase to said animal feed. A further aspect relates to a method of generating a prebiotic in-situ in a maize-based animal feed comprising the use of a GH30 glucuronoxylan hydrolase added to said feed. The invention is further directed to a method of improving the intestinal health of a monogastric animal comprising the administration of an enzyme-enriched maize-based animal feed to said animal wherein said animal feed comprises the enzyme GH30 glucuronoxylan hydrolase. As essential feature of the invention is the use of GH30 glucuronoxylan hydrolase in the methods of the invention. The methods, feeds or uses of the invention are typically wherein the GH30 glucuronoxylan hydrolase (EC 3.2.1.136) is a GH30_8 glucuronoxylan hydrolase.

The term “GH30 glucuronoxylan hydrolase” means a glucuronoarabinoxylan endo-1,4-beta-xylanase (E.C. 3.2.1.136) that catalyses the endohydrolysis of 1,4-beta-D-xylosyl links in some glucuronoarabinoxylans.

The term “wild-type” glucuronoxylan hydrolase means a glucuronoxylan hydrolase expressed by a naturally occurring microorganism, such as a bacterium, yeast, or filamentous fungus found in nature.

SEQ ID NO 1 is a “wild-type” glucuronoxylan hydrolase and is defined in W003106654 by SEQ ID NO:190. Nine single site amino acid mutations have also been prepared at positions D8F, QIIH, N12L, GI7I, G60H, P64V, S65V, G68A & S79P. Each of these mutations, alone or in combination, have improved thermal tolerance relative to the wild type enzyme (as measured following a heat challenge at 80° C. for 20 minutes). In one embodiment of the invention the methods of the invention comprise the use of a polypeptide having a glucuronoxylan hydrolase and
a) comprising a polypeptide having at least 80% sequence identity to any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 , SEQ ID NO:20, and SEQ ID NO:21;
b) comprising a polypeptide having at least 80% sequence identity to SEQ ID NO:1 and further comprising one or more mutations selected from the group comprising of D8F, QIIH, N12L, GI7I, G60H, P64V, S65V, G68A & S79P.

In a suitable embodiment, the polypeptide comprises at least one, such as at least two, such as at least three, such as at least four, such as at least five, such as at least six, such as at least seven, such as at least eight, such as nine mutations selected from the group comprising of D8F, QIIH, N12L, GI7I, G60H, P64V, S65V, G68A & S79P.

The polypeptide of the invention may be selected from the group consisting of
i. a polypeptide having at least 80% sequence identity, such as at least 85% sequence identity, such as at least 90% sequence identity, such as at least 95% sequence identity, such as at least 98% sequence identity, such as at least 99% sequence identity to SEQ ID NO 1;
ii. a polypeptide having at least 80% sequence identity, such as at least 85% sequence identity, such as at least 90% sequence identity, such as at least 95% sequence identity, such as at least 98% sequence identity, such as at least 99% sequence identity to SEQ ID NO 2; and
iii. a polypeptide having at least 80% sequence identity, such as at least 85% sequence identity, such as at least 90% sequence identity, such as at least 95% sequence identity, such as at least 98% sequence identity, such as at least 99% sequence identity to SEQ ID NO 3.

In a suitable embodiment, the GH30 glucuronoxylan hydrolase originates from Bacillus subtilis and wherein the GH30 glucuronoxylan hydrolase is a polypeptide have xylanase activity selected from the group consisting of
i. a polypeptide having at least 80% sequence identity, such as at least 85% sequence identity, such as at least 90% sequence identity, such as at least 95% sequence identity, such as at least 98% sequence identity, such as at least 99% sequence identity to SEQ ID NO 1;
ii. a polypeptide having at least 80% sequence identity, such as at least 85% sequence identity, such as at least 90% sequence identity, such as at least 95% sequence identity, such as at least 98% sequence identity, such as at least 99% sequence identity to SEQ ID NO 2; and
iii. a polypeptide having at least 80% sequence identity, such as at least 85% sequence identity, such as at least 90% sequence identity, such as at least 95% sequence identity, such as at least 98% sequence identity, such as at least 99% sequence identity to SEQ ID NO 3.
In a preferable embodiment, the GH30 glucuronoxylan hydrolase comprises, consists essentially of, or consists of SEQ ID NO 1, SEQ ID NO 2; or SEQ ID NO 3.

Improvement of the Feed Conversion Ratio

An aspect of the invention is directed to a method of improving the feed conversion ratio of an animal feed comprising maize comprising adding GH30 glucuronoxylan hydrolase to said animal feed. In a preferred embodiment, the feed is a maize-based or comprises maize. In one embodiment, the maize is fibrous maize. The feed may further comprise maize DDGS. In a still further embodiment, the feed may further comprise soybean meal.

An interesting aspect of the invention is directed to a method of improving the feed conversion ratio of monogastric animal comprising the use of GH30 glucuronoxylan hydrolase in a maize-based animal feed.

As can be seen from Example 1, body weight of poultry increased from an average of 421.2 g to 430.8 g after 14 days, from an average of 854.1 g to 893.75 g after 21 days (4,6%) and from an average of 1654.8 g to 1672.1 g after 28 days, all while reducing or at worst maintaining food intake. This represents substantial cost savings and increased revenue for a farmer.

The invention is also directed to the feed, namely an enzyme-enriched animal feed comprising GH30 glucuronoxylan hydrolase and maize, typically wherein the feed comprises maize in an amount of 100 to 1000 g/kg feed and GH30 glucuronoxylan hydrolase in an amount of 2 to 100 ppm per kg of feed.

A further aspect of the invention is directed to the use of GH30 glucuronoxylan hydrolase to prepare an enzyme-enriched animal feed, wherein said animal feed is a maize-based animal feed. A related aspect is directed to a method of improving the feed conversion ratio of monogastric animal comprising the use of GH30 glucuronoxylan hydrolase in a maize-based animal feed. The invention is directed in a similar aspect to the use of GH30 glucuronoxylan hydrolase to prepare an enzyme-enriched animal feed, wherein said animal feed is a maize-based animal feed. A maize-based feed is intended to mean a feed comprising maize in an amount of 100 to 1000 g/kg feed. Typically, the feed comprises maize in an amount of 100 to 1000 g/kg feed, such as 100 to 800 g/kg feed, such as 200 to 800 g/kg feed, such as 200 to 600 g/kg feed, such as 300 to 600 g/kg feed. Alternatively defined, in a maize-based feed, at least 10% wt/wt of the feed is maize or maize and maize DDGS, such as from 10% to 100%, from 10% to 80%, such as from 20% to 80%, such as from 25% to 85%, such as from 20% to 75%, from 25% to 75%, from 30% to 75%, typically from 30% to 70%, or 30% to 60%, suitably 35% to 65%.

In a typical embodiment of the feed, the enzyme-enriched animal feed comprises GH30 glucuronoxylan hydrolase and maize wherein the feed comprises maize in an amount of 100 to 1000 g/kg feed and GH30 glucuronoxylan hydrolase in an amount of 2 to 100 ppm per kg of feed; such as 100 to 800 g/kg feed and GH30 glucuronoxylan hydrolase in an amount of 2 to 100 ppm per kg of feed, such as 200 to 800 g/kg feed and GH30 glucuronoxylan hydrolase in an amount of 2 to 100 ppm per kg of feed, such as 200 to 600 g/kg feed and GH30 glucuronoxylan hydrolase in an amount of 2 to 100 ppm per kg of feed, such as 300 to 600 g/kg feed and GH30 glucuronoxylan hydrolase in an amount of 2 to 100 ppm per kg of feed.

The GH30 glucuronoxylan hydrolase may be present in the feed in an amount of 2 to 100 ppm per kg of feed, such as 2 to 80 ppm per kg, such as 2 to 60 ppm per kg, such as 2 to 50 ppm per kg, 2 to 40 ppm per kg, or 2 to 30 ppm per kg, or 2 to 20 ppm per kg. Suitably, the GH30 glucuronoxylan hydrolase may be present in the feed in an amount of 2 to 100 ppm per kg of feed, such as 5 to 80 ppm per kg, 10 to 60 ppm per kg, more typically 10 to 40 ppm per kg and 10 to 30 ppm per kg. The animal feed typically comprises 2 to 100 ppm of the GH30 glucuronoxylan hydrolase per kg of feed, such as 2 to 50 ppm, such as 2 to 40 ppm, such as 2.5 to 25 ppm, such as 5 to 20 ppm.

According to the methods of the invention, the animal is typically a monogastric animal, such as poultry or swine. The animal may be a chicken, such as a broiler chicken.

Improving the Intestinal Health

The present Examples also demonstrate that an endo-acting glucuronoxylan hydrolases from the family GH30 has a surprising solubilising effect on maize GAX into AXOS and its impact on broiler cecal microbiota composition in vitro.

The GH30 significantly increased (P<0.05) solubility of different insoluble NSP components from maize fibre. Oligomers solubilised by the GH30 contained a high amount of arabinose, reflected by the significant change in insoluble ara/xyl ratio by the GH30. Without being bound to a particular theory, the statistically significant increase in solubilized rhamnose and galactose may indicate pectin depolymerization as well while the significant increase (P<0.05) in glucose may stem from beta-glucans, also present in small amounts in maize grains.

Table 2 shows the in vitro SOFA production (mM) from 24 h and 48 h cecal fermentations of maize fibre at 37° C. using cecal broiler content as inoculum incubated without or with GH30 dosed at 10 ppm (n=3) as performed in Example 1. In vitro cecal fermentation of maize fibre supplemented with GH30 significantly increased (P<0.05) certain SOFA, in particular butyrate (table 2). As shown in Example 1 (Table 2), the method of the invention comprising the use of a GH30 in animal feed comprising maize fibre surprisingly resulted in an increase in butyrate formation by over 70%. The increase in butyrate formation is due to higher GAX solubilization by the GH30 of the invention (see Table 1). One aspect of the invention is directed to improving the intestinal health of a monogastric animal comprising adding a GH30 to animal feed. Alternatively defined, the method is directed to altering the microflora of a monogastric comprising the use of GH30 of the invention.

| Time point 24 h | | | 48 h | | Pooled |
|---|---|---|---|---|---|
| Group | Blank | GH30 | Blank | GH30 | SEM¹ |
| Acetate | 20.6^{b} | 41.2^{a} | 89.0^{b} | 107.6^{a} | 23.77 |
| Propionate | 8.1^{a} | 7.2^{a} | 15.0^{a} | 16.8^{a} | 5.78 |
| lsobutyrate | 1.4^{a} | 0.9^{a} | 3.8^{a} | 2.5^{b} | 0.41 |
| Butyrate | 3.2^{b} | 4.9^{a} | 5.3^{b} | 9.1^{a} | 0.96 |
| lsovalerate | 2.9^{a} | 2.1^{a} | 6.6^{a} | 4.5^{b} | 0.75 |
| Valerate | 0.6^{a} | 0.2^{b} | 11.2^{a} | 5.3^{b} | 1.10 |
| Total SCFA | 36.6^{a} | 56.4^{a} | 130.9^{b} | 145.7^{a} | 25.22 |

¹SEM = Standard error of mean summation of both 24 h and 48 h.
^{abc}Mean values within a column not sharing a common letter differ significantly (P < 0.05; Tukey-Kramer HSD) (n = 3).

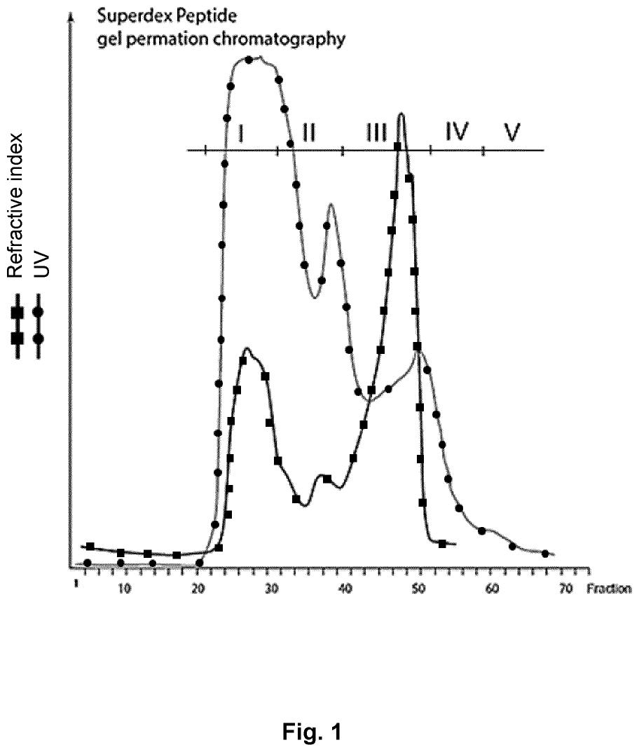

Oligosaccharides are defined as saccharides containing between 3 and 10 sugar moieties. To test the oligosaccharide and polysaccharide size (MW) solubilised by a xylanase on the cecal microbiome, AXOS generated by the GH30 was separated by SEC into 4 Pools (I, II, Ill, IV) until ˜100 mg material (freeze dried) of each fraction was obtained. During the separation of the oligosaccharides generated by the GH30, the UV signal (280 nm) characteristically followed the RI signal indicating that oligomers in the different fractions contained aromatic compounds. The signal stems from ferulic acid (the main source of phenolics in xylans) with coumaric acid or other phenolic compounds in small amounts (Boz, 2015; Bunzel, 2010). SEC of a control supernatant (without GH30 added) showed low or no UV signal and a corresponding low RI signal (data not shown). It is clear that the highest amount of AXOS present in the supernatant from maize fibre incubated with GH30 with an average MW of 1000-4000 Da (average degree of polymerization of approximately 7-40 pentose units) was in pool III, indicated by the peak in RI (FIG. 1). However, the highest amount of total SOFA along with butyrate was observed in pool II with an average MW of 4000-10000 Da (average degree of polymerization of approximately 40-76) at both 24 h (significantly higher) and 48 h (numerically higher) indicating that the cecal microbiome may prefer longer chains of AXOS during the formation of butyrate, which has also been shown to be beneficial in broilers in the case of wheat-based AXOS.

The bacterial composition changed significantly with pool IV, where growth of Ruminococcaceae and Lachnospiraceae families, including genus Faecalibacterium, were significantly increased (P<0.05), while Bacteroides were significantly lowered (P<0.05) compared to the other pools containing shorter oligo- and polysaccharides, as seen in FIG. 1 However, a more specific and precise separation of polysaccharide and oligosaccharide may be needed for a more detailed investigation of the bacterial composition change.

Differential abundance analysis with Deseq2 revealed a Bacteroides species (OTU3), which was 99% identical to Bacteroides xylanisolvens, was significantly decreased (P<0.05) with GH30 supplementation. Bacteroides xylanisolvens is known for its vast repertoire of genes targeted at xylan utilization. Bacteroides xylanisolvens prefers degradation of long soluble polysaccharides. The observed reduction in growth of Bacteroides xylanisolvens is due to the addition of the exogenous GH30 glucuronoxylan hydrolase of the invention, as the endogenous genes involved in long xylan degradation are less needed. An increase in diversity was observed with addition of GH30. High microbial diversity is recognized to be directly linked to intestinal health. Accordingly, the invention is further directed to a method of increasing microbial diversity in a monogastric animal.

The solubilisation of GAX had a minimal effect on Firmicutes phylum level, however it appeared to favour butyrate-producing bacteria, such as OTU11, classified as Faecalibacterium which is one of the most abundant butyrate-producing bacteria in monogastric animals. Furthermore, bacteria from families Lachnospiraceae and Ruminococcaceae were also significantly increased (P<0.05) in samples treated with GH30. Lachnospiraceae are anaerobes and many species are associated with the production of butyrate, which is beneficial for both host epithelial cell and microbiota growth. This corresponds well with the observed significant increase in butyrate in the in vitro fermentations. Previous studies also show higher butyrate levels with addition of GH11 xylanase to xylan-rich fibre substrates such as wheat bran accompanied by a reduction in Bacteroides spp. It has been found that Bacteroides xylanisolvens is unable to break down starch (Chassard et al., 2008) whereas Bacteroides vulgatus is a known starch degrader (McCarthy et al., 1988). The addition of GH30 to the fermentation media will likely make the leftover starch more available and consequently favour the growth of the starch utilizing Bacteroides vulgatus over the non-starch utilizer Bacteroides xylanisolvens. Moreover, Bacteroides vulgatus is known for pectin depolymerisation and degradation of the galactose released from the pectins (Hobbs et al., 2014). A significant increase (P<0.001) in Bifidobacterium with GH30 supplementation was also observed. In humans, studies have shown an association between Bifidobacterium and wheat-derived AXOS with health benefits, where bifidogenic properties abolished metabolic disorders induced by a western diet in mice. A method of the invention is directed to increasing the intestinal levels of bytyrate producing bacteria, such as bacteria selected from the group consisting of Bifidobacterium, Ruminococcaceae (including genus Faecalibacterium) and Lachnospiraceae, in monogastrics.

The method of the invention provides for an understanding fibre metabolism in monogastric animals and identifying beneficial commensal bacteria groups and how to affect their growth. Accordingly, the method of the invention is important in optimising animal gut health. Solubilisation of insoluble maize fibre fractions with an exogenous GH30 glucuronoxylan hydrolase acting in situ according to the invention allows for an increase in fibre fermentability, contributes to increase microbial diversity, and promotes beneficial bacterial shifts in the cecal broiler microbiota. The Examples show a clear Bacteroides reduction effect by an exogenous glucuronoxylan hydrolase with specificity towards maize GAX, while bacterial families Lachnospiraceae and Ruminococcaceae (including genus Faecalibacterium) which are critically important butyrate producers were significantly increased. These bacteria caused a butyrogenic effect during fermentation, provides a health benefit of a maize GAX degrading enzyme.

The Examples of the present disclosure further reveal a novel mode of action of GH30 feed enzymes leading to a new field of use of the GH30 enzymes. The impact on the intestinal environment and microbiota is an aspect yet unknown to GH30s in feed enzymes allowing for improvement of the gut health in animals. As shown in the Examples, the enzymatic breakdown products generated in situ in the intestine was analysed and the effect on broiler gut morphology and microbiota composition was investigated. NSP analysis and confocal microscopy of the jejunum digesta showed the maize GAX solubilisation effect by the hydrolase. The GH30 targeted and lowered (P<0.05) the insoluble part of the GAX. The solubilised AXOS have a high ara/xyl substitution degree as demonstrated by the amount of arabinose (15.9%) compared to xylose (15.7%) solubilised from the insoluble NSP fraction after acid hydrolysis.

The NSP analysis showed a small increase (P<0.05) in solubilised galactose and rhamnose in the jejunum digesta found upon GH30 supplementation. Without being bound to a particular theory, this increase is thought to be due to the solubilisation of the pectic polysaccharide rhamnogalacturonan-I (RG-I), also present in maize cell walls wherein the RG-I then acts as a prebiotic. The improved animal performance observed in the Examples is due to the solubilisation of glucurono-arabinoxylan from the maize and the maize DDGS. The GH30 cleaves and solubilises the highly branched and heterogenous glucurono-arabinoxylan structure and the higher level of soluble arabinoxylooligosaccharide yielded more energy from increased hindgut microbial fermentation and/or higher absorption and accessibility of nutrients.

As shown in the Examples, butyrate levels in the ceca of GH30 supplemented birds increased (P<0.05) and there was a general tendency for higher acetate and total SOFA levels as well (Table 3).

TABLE 3
SCFA concentrations (μmol/g) in cecal content of broilers
supplemented without and with dietary GH30 (n = 24)

| Group | Control | GH30 | Pooled SEM¹ |
|---|---|---|---|
| Acetate | 51.31^{a} | 55.74^{a} | 4.19 |
| Propionate | 9.21^{a} | 9.23^{a} | 0.91 |
| lsobutyrate | 0.66^{a} | 0.55^{a} | 0.09 |
| Butyrate | 10.92^{b} | 14.49^{a} | 1.74 |
| lsovalerate | 1.28^{a} | 1.38^{a} | 0.17 |
| Valerate | 1.22^{a} | 1.32^{a} | 0.13 |
| Total SCFA | 75.99^{a} | 82.72^{a} | 6.17 |

¹SEM = Standard Error Mean.
^{ab}Mean values within a column not sharing a common letter differ significantly (P < 0.05; Tukey-Kramer HSD) (n = 24).

Likewise, during in vitro fermentation with cecal microbiota, a numerical increase in total SOFA was observed with addition of a GH30 to maize fibre (Table 4). The in vitro fermentations showed that some of the soluble arabinoxylooligosaccharide generated by the GH30 were butyrogenic yielding 43.6% more butyrate compared to control and an apparent shift from propionate and valerate to butyrate (Table 4). Stimulating the colonization and growth of butyrate-producing bacteria optimizes gut health. Accordingly, one aspect of the invention is directed to a method of improving gut health of a monogastric animal comprising the use of a GH30 glucuronoxylan hydrolase. Higher levels of butyrate have beneficial effects on gut morphology and stimulates mucin glycoproteins expression in intestinal epithelial cells. An increase (P<0.0038) in the ratio of butyryl-CoA:acetate-CoA transferase gene to total bacteria in birds supplemented with the GH30 was observed. This at least in part explains the reduction (P<0.001) in T-lymphocyte infiltration and increase (P<0.001) in villi length in the duodenum, since butyrate is known to reduce inflammation and increase epithelial cell multiplication and differentiation.

TABLE 4
In vitro SCFA production (mM) from 6 h, 24 h and 48 h cecal fermentations of maize
fibre containing 110 g/kg xylose at 37° C. using cecal broiler content as inoculum
incubated without or with SEQ ID NO 1 dosed at 10 ppm (n = 3)

| | Time point | | | | | | |
|---|---|---|---|---|---|---|---|
| | 5 h | | 24 h | | 48 h | | |
| | | SEQ ID | | SEQ ID | | SEQ ID | Pooled |
| Group | Control | NO 1 | Control | NO 1 | Control | NO 1 | SEM¹ |
| Acetate | 16.91^{a} | 24.01^{a} | 19.92^{a} | 38.19^{a} | 88.67^{a} | 107.11^{a} | 26.22 |
| Propionate | 5.44^{a} | 3.47^{a} | 7.99^{a} | 6.98^{a} | 24.72^{a} | 16.59^{b} | 5.27 |
| Isobutyrate | 0.42^{a} | 0.19^{a} | 1.36^{a} | 0.88^{a} | 3.72^{a} | 2.74^{a} | 0.80 |
| Butyrate | 0.96^{a} | 0.61^{b} | 3.13^{b} | 4.24^{ab} | 5.30^{b} | 9.40^{a} | 1.53 |
| Isovalerate | 0.43^{a} | 0.20^{b} | 2.83^{a} | 2.06^{a} | 6.59^{a} | 4.58^{a} | 1.3 |
| Valerate | 0.41^{a} | 0.10^{a} | 0.62^{ab} | 0.19^{b} | 11.00^{a} | 5.02^{b} | 0.87 |
| Total SCFA | 24.58^{a} | 28.67^{a} | 35.84^{a} | 52.54^{a} | 139.99^{a} | 145.45^{a} | |

¹SEM = Standard error of mean.
^{ab}Mean values within a column not sharing a common letter differ significantly
(P < 0.05; Tukey-Kramer HSD)
(n = 3).

An aspect of the invention is directed to a method of generating a prebiotic in-situ in a maize-based animal feed comprising the use of a GH30 glucuronoxylan hydrolase added to said feed. At least one of the prebiotics that is generated in-situ is an arabinoxylan oligosaccharide and polysaccharides. An aspect of the invention is a method of decreasing the insoluble maize fraction in a maize-based animal feed comprising the addition of a GH30 glucuronoxylan hydrolase.

A further aspect of the invention is directed to a method of improving the intestinal health of a monogastric animal comprising the administration of an enzyme-enriched maize-based animal feed to said animal wherein said animal feed comprises the enzyme GH30 glucuronoxylan hydrolase. Typically, the GH30 glucuronoxylan hydrolase degrades the non-starch polysaccharides of said maize so as to generate prebiotic oligomers and polymers, prebiotic oligomers and polymers comprising arabinoxylan oligosaccharides. Accordingly, an alternative aspect of the invention is a method of improving the intestinal health of a monogastric animal by in-situ production of arabinoxylan oligosaccharides and polysaccharides. An alternative method of the invention, is a method for the in-situ production of prebiotics in monogastric animals comprising the administration of an enzyme-enriched maize-based animal feed to said animal wherein said animal feed comprises the enzyme GH30 glucuronoxylan hydrolase. Alternatively defined, an aspect of the invention is directed to a method for improving intestinal health in a monogastic animal, said method comprising increasing the levels of cecal butyrate levels in situ in said animal said method comprising the administration of an enzyme-enriched maize-based animal feed to said animal wherein said animal feed comprises the enzyme GH30 glucuronoxylan hydrolase.

The invention is furthermore directed to a method for improving intestinal health in a monogastic animal said method comprising altering the microbiota composition in said animal by administration of an enzyme-enriched maize-based animal feed to said animal wherein said animal feed comprises the enzyme GH30 glucuronoxylan hydrolase. As explained above, the microbiota composition of said animal is typically altered in that, at least, Baceteroide levels are reduced and Lachnospiraceae and/or Ruminococcaceae levels are increased.

A further aspect of the invention is directed to a method of causing a butyrogenic effect in a monogastic animal comprising the administration of an enzyme-enriched maize-based animal feed to said animal wherein said animal feed comprises the enzyme GH30 glucuronoxylan hydrolase.

Enzyme Compositions

The present invention also relates to compositions comprising a polypeptide of the present invention. Preferably, the compositions are enriched in the polypeptide of the invention. The term “enriched” indicates that the GH30 glucuronoxylan hydrolase activity of the composition has been increased, e.g., with an enrichment factor of at least 1.1, such as at least 1.2, at least 1.3, at least 1.4, at least 1.5, at least 2.0, at least 3.0, at least 4.0, at least 5.0, at least 10.

In an embodiment, the composition comprises one or more polypeptides of the invention and one or more formulating agents, as described below.

The compositions may further comprise multiple enzymatic activities, such as one or more (e.g., several) enzymes selected from the group consisting of phytase, xylanase, galactanase, protease, phospholipase A1, phospholipase A2, lysophospholipase, phospholipase C, phospholipase D, amylase, lysozyme, arabinofuranosidase, beta-xylosidase, acetyl xylan esterase, feruloyl esterase, cellulase, cellobiohydrolases, beta-glucosidase, pullulanase, and beta-glucanase or any combination thereof.

The compositions may further comprise one or more microbes. In an embodiment, the microbe is selected from the group consisting of Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Bifidobacterium bifidum, Bifidobacterium animalis, Bifidobacterium sp., Carnobacterium sp., Clostridium butyricum, Clostridium sp., Enterococcus faecium, Enterococcus sp., Lactobacillus sp., Lactobacillus acidophilus, Lactobacillus farciminus, Lactobacillus rhamnosus, Lactobacillus reuteri, Lactobacillus salivarius, Lactococcus lactis, Lactococcus sp., Leuconostoc sp., Megasphaera elsdenii, Megasphaera sp., Pediococsus acidilactici, Pediococcus sp., Propionibacterium thoenii, Propionibacterium sp. and Streptococcus sp. or any combination thereof.

Formulating Agent

The enzyme of the invention may be formulated as a liquid or a solid. For a liquid formulation, the formulating agent may comprise a polyol (such as e.g. glycerol, ethylene glycol or propylene glycol), a salt (such as e.g. sodium chloride, sodium benzoate, potassium sorbate) or a sugar or sugar derivative (such as e.g. dextrin, glucose, sucrose, and sorbitol). Thus, in one embodiment, the composition is a liquid composition comprising the polypeptide of the invention and one or more formulating agents selected from the list consisting of glycerol, ethylene glycol, 1,2-propylene glycol, 1,3-propylene glycol, sodium chloride, sodium benzoate, potassium sorbate, dextrin, glucose, sucrose, and sorbitol. The liquid formulation may be sprayed onto the feed after it has been pelleted or may be added to drinking water given to the animals.

For a solid formulation, the formulation may be for example as a granule, spray dried powder or agglomerate. The formulating agent may comprise a salt (organic or inorganic zinc, sodium, potassium or calcium salts such as e.g. such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol).

In an embodiment, the solid composition is in granulated form. The granule may have a matrix structure where the components are mixed homogeneously. However, the granule typically comprises a core particle and one or more coatings, which typically are salt and/or wax coatings. Examples of waxes are polyethylene glycols; polypropylenes; Carnauba wax; Candelilla wax; bees wax; hydrogenated plant oil or animal tallow such as hydrogenated ox tallow, hydrogenated palm oil, hydrogenated cotton seeds and/or hydrogenated soy bean oil; fatty acid alcohols; mono-glycerides and/or di-glycerides, such as glyceryl stearate, wherein stearate is a mixture of stearic and palmitic acid; micro-crystalline wax; paraffin's; and fatty acids, such as hydrogenated linear long chained fatty acids and derivatives thereof. A preferred wax is palm oil or hydrogenated palm oil. The core particle can either be a homogeneous blend of GH30 glucuronoxylan hydrolase of the invention optionally combined with one or more additional enzymes and optionally together with one or more salts or an inert particle with the GH30 glucuronoxylan hydrolase of the invention optionally combined with one or more additional enzymes applied onto it.

In an embodiment, the material of the core particles are selected from the group consisting of inorganic salts (such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol), sugar or sugar derivative (such as e.g. sucrose, dextrin, glucose, lactose, sorbitol), small organic molecules, starch, flour, cellulose and minerals and clay minerals (also known as hydrous aluminium phyllosilicates). In a preferred embodiment, the core comprises a clay mineral such as kaolinite or kaolin.

The salt coating is typically at least 1 μm thick and can either be one particular salt or a mixture of salts, such as Na₂SO₄, K₂SO₄, MgSO₄ and/or sodium citrate. Other examples are those described in e.g. WO 2008/017659, WO 2006/034710, WO 1997/05245, WO 1998/54980, WO 1998/55599, WO 2000/70034 or polymer coating such as described in WO 2001/00042.

In another embodiment, the composition is a solid composition comprising the GH30 glucuronoxylan hydrolase of the invention and one or more formulating agents selected from the list consisting of sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch and cellulose. In a preferred embodiment, the formulating agent is selected from one or more of the following compounds: sodium sulfate, dextrin, cellulose, sodium thiosulfate and calcium carbonate. In a preferred embodiment, the solid composition is in granulated form. In an embodiment, the solid composition is in granulated form and comprises a core particle, an enzyme layer comprising the GH30 glucuronoxylan hydrolase of the invention and a salt coating.

In a further embodiment, the formulating agent is selected from one or more of the following compounds: glycerol, ethylene glycol, 1,2-propylene glycol or 1,3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, kaolin and cellulose. In a preferred embodiment, the formulating agent is selected from one or more of the following compounds: 1,2-propylene glycol, 1,3-propylene glycol, sodium sulfate, dextrin, cellulose, sodium thiosulfate, kaolin and calcium carbonate.

Formulating Agent

The enzyme of the invention may be formulated as a liquid or a solid or a semi-solid formulation. For a liquid formulation, the formulating agent may comprise a polyol (such as e.g. glycerol, ethylene glycol or propylene glycol), a salt (such as e.g. sodium chloride, sodium benzoate, potassium sorbate) or a sugar or sugar derivative (such as e.g. dextrin, glucose, sucrose, and sorbitol). Thus, in one embodiment, the composition is a liquid composition comprising the polypeptide of the invention and one or more formulating agents selected from the list consisting of glycerol, ethylene glycol, 1,2-propylene glycol, 1,3-propylene glycol, sodium chloride, sodium benzoate, potassium sorbate, dextrin, glucose, sucrose, and sorbitol.

For a solid formulation, the formulation may be for example as a granule, spray dried powder or agglomerate. The formulating agent may comprise a salt (organic or inorganic zinc, sodium, potassium or calcium salts such as e.g. such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol).

In an embodiment, the solid composition is in granulated form. The granule may have a matrix structure where the components are mixed homogeneously. However, the granule typically comprises a core particle and one or more coatings, which typically are salt and/or wax coatings. The core particle can either be a homogeneous blend of GH30 glucuronoxylan hydrolase of the invention optionally combined with one or more additional enzymes and optionally together with one or more salts or an inert particle with the GH30 glucuronoxylan hydrolase of the invention optionally combined with one or more additional enzymes applied onto it.

In an embodiment, the material of the core particles are selected from the group consisting of inorganic salts (such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol), sugar or sugar derivative (such as e.g. sucrose, dextrin, glucose, lactose, sorbitol), small organic molecules, starch, flour, cellulose and minerals.

The salt coating is typically at least 1 pm thick and can either be one particular salt or a mixture of salts, such as Na₂SO₄, K₂SO₄, MgSO₄ and/or sodium citrate. Other examples are those described in e.g. WO 2008/017659, WO 2006/034710, WO 1997/05245, WO 1998/54980, WO 1998/55599, WO 2000/70034 or polymer coating such as described in WO 2001/00042.

In another embodiment, the composition is a solid composition comprising the xylanase of the invention and one or more formulating agents selected from the list consisting of sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch and cellulose. In a preferred embodiment, the formulating agent is selected from one or more of the following compounds: sodium sulfate, dextrin, cellulose, sodium thiosulfate and calcium carbonate. In a preferred embodiment, the solid composition is in granulated form. In an embodiment, the solid composition is in granulated form and comprises a core particle, an enzyme layer comprising the GH30 glucuronoxylan hydrolase of the invention and a salt coating.

In a further embodiment, the formulating agent is selected from one or more of the following compounds: glycerol, ethylene glycol, 1,2-propylene glycol or 1,3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch and cellulose. In a preferred embodiment, the formulating agent is selected from one or more of the following compounds: 1,2-propylene glycol, 1,3-propylene glycol, sodium sulfate, dextrin, cellulose, sodium thiosulfate and calcium carbonate.

Animal Feed and Animal Feed Additives

The present invention also relates to animal feed compositions and animal feed additives comprising one or more GH30 glucuronoxylan hydrolases of the invention. In an embodiment, the animal feed or animal feed additive comprises a formulating agent and one or more GH30 glucuronoxylan hydrolases of the invention. In a further embodiment, the formulating agent comprises one or more of the following compounds: glycerol, ethylene glycol, 1,2-propylene glycol or 1,3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, kaolin and cellulose.

Animal feed compositions or diets have a relatively high content of protein. Poultry and pig diets can be characterised as indicated in Table B of WO 01/58275, columns 2-3. Fish diets can be characterised as indicated in column 4 of this Table B. Furthermore, such fish diets usually have a crude fat content of 200-310 g/kg.

An animal feed composition according to the invention has a crude protein content of 50-800 g/kg, and furthermore comprises at least one GH30 glucuronoxylan hydrolase as claimed herein.

Furthermore, or in the alternative (to the crude protein content indicated above), the animal feed composition of the invention has a content of metabolisable energy of 10-30 MJ/kg; and/or a content of calcium of 0.1-200 g/kg; and/or a content of available phosphorus of 0.1-200 g/kg; and/or a content of methionine of 0.1-100 g/kg; and/or a content of methionine plus cysteine of 0.1-150 g/kg; and/or a content of lysine of 0.5-50 g/kg.

In particular embodiments, the content of metabolisable energy, crude protein, calcium, phosphorus, methionine, methionine plus cysteine, and/or lysine is within any one of ranges 2, 3, 4 or 5 in Table B of WO 01/58275 (R. 2-5).

Crude protein is calculated as nitrogen (N) multiplied by a factor 6.25, i.e. Crude protein (g/kg)=N (g/kg)×6.25. The nitrogen content is determined by the Kjeldahl method (A.O.A.C., 1984, Official Methods of Analysis 14th ed., Association of Official Analytical Chemists, Washington D.C.).

Metabolisable energy can be calculated on the basis of the NRC publication Nutrient requirements in swine, ninth revised edition 1988, subcommittee on swine nutrition, committee on animal nutrition, board of agriculture, national research council. National Academy Press, Washington, D.C., pp. 2-6, and the European Table of Energy Values for Poultry Feed-stuffs, Spelderholt centre for poultry research and extension, 7361 DA Beekbergen, The Netherlands. Grafisch bedrijf Ponsen & looijen by, Wageningen. ISBN 90-71463-12-5.

The dietary content of calcium, available phosphorus and amino acids in complete animal diets is calculated on the basis of feed tables such as Veevoedertabel 1997, gegevens over chemische samenstelling, verteerbaarheid en voederwaarde van voedermiddelen, Central Veevoederbureau, Runderweg 6, 8219 pk Lelystad. ISBN 90-72839-13-7.

In a particular embodiment, the animal feed composition of the invention contains at least one vegetable protein as defined above.

The animal feed composition of the invention may also contain animal protein, such as Meat and Bone Meal, Feather meal, and/or Fish Meal, typically in an amount of 0-25%. The animal feed composition of the invention may also comprise Dried Distillers Grains with Solubles (DDGS), typically in amounts of 0-30%.

In still further particular embodiments, the animal feed composition of the invention contains 0-80% maize; and/or 0-80% sorghum; and/or 0-70% wheat; and/or 0-70% Barley; and/or 0-30% oats; and/or 0-40% soybean meal; and/or 0-25% fish meal; and/or 0-25% meat and bone meal; and/or 0-20% whey.

The animal feed may comprise vegetable proteins. In particular embodiments, the protein content of the vegetable proteins is at least 10, 20, 30, 40, 50, 60, 70, 80, or 90% (w/w). Vegetable proteins may be derived from vegetable protein sources, such as legumes and cereals, for example, materials from plants of the families Fabaceae (Leguminosae), Cruciferaceae, Chenopodiaceae, and Poaceae, such as soy bean meal, lupin meal, rapeseed meal, and combinations thereof.

In a particular embodiment, the vegetable protein source is material from one or more plants of the family Fabaceae, e.g., soybean, lupine, pea, or bean. In another particular embodiment, the vegetable protein source is material from one or more plants of the family Chenopodiaceae, e.g. beet, sugar beet, spinach or quinoa. Other examples of vegetable protein sources are rapeseed, and cabbage. In another particular embodiment, soybean is a preferred vegetable protein source. Other examples of vegetable protein sources are cereals such as barley, wheat, rye, oat, maize (corn), rice, and sorghum.

Animal diets can e.g. be manufactured as mash feed (non-pelleted) or pelleted feed. Typically, the milled feed-stuffs are mixed and sufficient amounts of essential vitamins and minerals are added according to the specifications for the species in question. Enzymes can be added as solid or liquid enzyme formulations. For example, for mash feed a solid or liquid enzyme formulation may be added before or during the ingredient mixing step. For pelleted feed the (liquid or solid) GH30 glucuronoxylan hydrolase/enzyme preparation may also be added before or during the feed ingredient step. Typically a liquid GH30 glucuronoxylan hydrolase/enzyme preparation comprises the GH30 glucuronoxylan hydrolase of the invention optionally with a polyol, such as glycerol, ethylene glycol or propylene glycol, and is added after the pelleting step, such as by spraying the liquid formulation onto the pellets. The enzyme may also be incorporated in a feed additive or premix.

Alternatively, the GH30 glucuronoxylan hydrolase can be prepared by freezing a mixture of liquid enzyme solution with a bulking agent such as ground soybean meal, and then lyophilizing the mixture.

In an embodiment, the animal feed or animal feed additive comprises one or more additional enzymes. In an embodiment, the animal feed comprises one or more microbes. In an embodiment, the animal feed comprises one or more vitamins. In an embodiment, the animal feed comprises one or more minerals. In an embodiment, the animal feed comprises one or more amino acids. In an embodiment, the animal feed comprises one or more other feed ingredients.

In another embodiment, the animal feed or animal feed additive comprises the polypeptide of the invention, one or more formulating agents and one or more additional enzymes. In an embodiment, the animal feed or animal feed additive comprises the polypeptide of the invention, one or more formulating agents and one or more microbes. In an embodiment, the animal feed comprises the polypeptide of the invention, one or more formulating agents and one or more vitamins. In an embodiment, the animal feed or animal feed additive comprises one or more minerals. In an embodiment, the animal feed or animal feed additive comprises the polypeptide of the invention, one or more formulating agents and one or more amino acids. In an embodiment, the animal feed or animal feed additive comprises the polypeptide of the invention, one or more formulating agents and one or more other feed ingredients.

In a further embodiment, the animal feed or animal feed additive comprises the polypeptide of the invention, one or more formulating agents and one or more components selected from the list consisting of: one or more additional enzymes; one or more microbes; one or more vitamins; one or more minerals; one or more amino acids; and one or more other feed ingredients.

Additional Enzymes

In another embodiment, the compositions described herein optionally include one or more enzymes. Enzymes can be classified on the basis of the handbook Enzyme Nomenclature from NC-IUBMB, 1992), see also the ENZYME site at the internet: http://www.expasy.ch/enzyme/. ENZYME is a repository of information relative to the nomenclature of enzymes. It is primarily based on the recommendations of the Nomenclature Committee of the International Union of Biochemistry and Molecular Biology (IUB-MB), Academic Press, Inc., 1992, and it describes each type of characterized enzyme for which an EC (Enzyme Commission) number has been provided (Bairoch A. The ENZYME database, 2000, Nucleic Acids Res 28:304-305). This IUB-MB Enzyme nomenclature is based on their substrate specificity and occasionally on their molecular mechanism; such a classification does not reflect the structural features of these enzymes.

Another classification of certain glycoside hydrolase enzymes, such as endoglucanase, alpha-galactosidase, galactanase, mannanase, dextranase, lysozyme and galactosidase is described in Henrissat et al, “The carbohydrate-active enzymes database (CAZy) in 2013”, Nucl. Acids Res. (1 Jan. 2014) 42 (D1): D490-D495; see also www.cazy.org.

Thus the composition of the invention may also comprise at least one other enzyme selected from the group comprising of phytase (EC 3.1.3.8 or 3.1.3.26); xylanase (EC 3.2.1.8); galactanase (EC 3.2.1.89); alpha-galactosidase (EC 3.2.1.22); protease (EC 3.4); phospholipase Al (EC 3.1.1.32); phospholipase A2 (EC 3.1.1.4); lysophospholipase (EC 3.1.1.5); phospholipase C (3.1.4.3); phospholipase D (EC 3.1.4.4); amylase such as, for example, alpha-amylase (EC 3.2.1.1); arabinofuranosidase (EC 3.2.1.55); beta-xylosidase (EC 3.2.1.37); acetyl xylan esterase (EC 3.1.1.72); feruloyl esterase (EC 3.1.1.73); cellulase (EC 3.2.1.4); cellobiohydrolases (EC 3.2.1.91); beta-glucosidase (EC 3.2.1.21); pullulanase (EC 3.2.1.41), alpha-mannosidase (EC 3.2.1.24), mannanase (EC 3.2.1.25) and beta-glucanase (EC 3.2.1.4 or EC 3.2.1.6), or any mixture thereof.

In a particular embodiment, the composition of the invention comprises a phytase (EC 3.1.3.8 or 3.1.3.26). Examples of commercially available phytases include Bio-Feed™ Phytase (Novozymes), Ronozyme® P, Ronozyme® NP and Ronozyme® HiPhos (DSM Nutritional Products), Natuphos® and Naturphos® E (BASF), Finase® and Quantum® Blue (AB Enzymes), OptiPhos® (Huvepharma) Phyzyme® XP (Verenium/DuPont) and Axtra® PHY (DuPont). Other preferred phytases include those described in e.g. WO 98/28408, WO 00/43503, and WO 03/066847.

In a particular embodiment, the composition of the invention comprises a GH30 glucuronoxylan hydrolase (EC 3.2.1.8). Examples of commercially available xylanases include Ronozyme® WX and Ronozyme® G2 (DSM Nutritional Products), Econase® XT and Barley (AB Vista), Xylathin® (Verenium), Hostazym® X (Huvepharma) and Axtra® XB (xylanase /beta-glucanase, DuPont).

In a particular embodiment, the composition of the invention comprises a protease (EC 3.4). Examples of commercially available proteases include Ronozyme® ProAct (DSM Nutritional Products).

Microbes

In an embodiment, the animal feed composition further comprises one or more additional microbes. In a particular embodiment, the animal feed composition further comprises a bacterium from one or more of the following genera: Lactobacillus, Lactococcus, Streptococcus, Bacillus, Pediococcus, Enterococcus, Leuconostoc, Carnobacterium, Propionibacterium, Bifidobacterium, Clostridium and Megasphaera or any combination thereof.

In a preferred embodiment, animal feed composition further comprises a bacterium from one or more of the following strains: Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Enterococcus faecium, Enterococcus spp, and Pediococcus spp, Lactobacillus spp, Bifidobacterium spp, Lactobacillus acidophilus, Pediococsus acidilactici, Lactococcus lactis, Bifidobacterium bifidum, Propionibacterium thoenii, Lactobacillus farciminus, lactobacillus rhamnosus, Clostridium butyricum, Bifidobacterium animalis ssp. animalis, Lactobacillus reuteri, Lactobacillus salivarius ssp. salivarius, Megasphaera elsdenii, Propionibacteria sp.

In a more preferred embodiment, animal feed composition further comprises a bacterium from one or more of the following strains of Bacillus subtilis: 3A-P4 (PTA-6506); 15A-P4 (PTA-6507); 22C-P1 (PTA-6508); 2084 (NRRL B-500130); LSSA01 (NRRL-B-50104); BS27 (NRRL B-501 05); BS 18 (NRRL B-50633); and BS 278 (NRRL B-50634).

The bacterial count of each of the bacterial strains in the animal feed composition is between 1×10⁴ and 1×10¹⁴ CFU/kg of dry matter, preferably between 1×10⁶ and 1×10¹² CFU/kg of dry matter, and more preferably between 1×10⁷ and 1×10¹¹ CFU/kg of dry matter. In a more preferred embodiment the bacterial count of each of the bacterial strains in the animal feed composition is between 1×10⁸ and 1×10¹⁰ CFU/kg of dry matter.

The bacterial count of each of the bacterial strains in the animal feed composition is between 1×10⁵ and 1×10¹⁵ CFU/animal/day, preferably between 1×10⁷ and 1×10¹³ CFU/animal/day, and more preferably between 1×10⁸ and 1×10¹² CFU/animal/day. In a more preferred embodiment the bacterial count of each of the bacterial strains in the animal feed composition is between 1×10⁹ and 1×10¹¹ CFU/animal/day.

In another embodiment, the one or more bacterial strains are present in the form of a stable spore.

Premix

In an embodiment, the animal feed may include a premix, comprising e.g. vitamins, minerals, enzymes, amino acids, preservatives, antibiotics, other feed ingredients or any combination thereof which are mixed into the animal feed.

Amino Acids

The composition of the invention may further comprise one or more amino acids. Examples of amino acids which are used in animal feed are lysine, alanine, beta-alanine, threonine, methionine and tryptophan.

Vitamins and Minerals

In another embodiment, the animal feed may include one or more vitamins, such as one or more fat-soluble vitamins and/or one or more water-soluble vitamins. In another embodiment, the animal feed may optionally include one or more minerals, such as one or more trace minerals and/or one or more macro minerals.

Usually fat- and water-soluble vitamins, as well as trace minerals form part of a so-called premix intended for addition to the feed, whereas macro minerals are usually separately added to the feed.

Non-limiting examples of fat-soluble vitamins include vitamin A, vitamin D3, vitamin E, and vitamin K, e.g., vitamin K3.

Non-limiting examples of water-soluble vitamins include vitamin B12, biotin and choline, vitamin B1, vitamin B2, vitamin B6, niacin, folic acid and panthothenate, e.g., Ca-D-panthothenate.

Non-limiting examples of trace minerals include boron, cobalt, chloride, chromium, copper, fluoride, iodine, iron, manganese, molybdenum, selenium and zinc.

Non-limiting examples of macro minerals include calcium, magnesium, potassium and sodium.

The nutritional requirements of these components (exemplified with poultry and piglets/pigs) are listed in Table A of WO 01/58275. Nutritional requirement means that these components should be provided in the diet in the concentrations indicated.

In the alternative, the animal feed additive of the invention comprises at least one of the individual components specified in Table A of WO 01/58275. At least one means either of, one or more of, one, or two, or three, or four and so forth up to all thirteen, or up to all fifteen individual components. More specifically, this at least one individual component is included in the additive of the invention in such an amount as to provide an in-feed-concentration within the range indicated in column four, or column five, or column six of Table A.

In a still further embodiment, the animal feed additive of the invention comprises at least one of the below vitamins, preferably to provide an in-feed-concentration within the ranges specified in the below Table 10 (for piglet diets, and broiler diets, respectively).

TABLE 10
Typical vitamin recommendations

| Vitamin | Piglet diet | | Broiler diet | |
|---|---|---|---|---|
| Vitamin A | 10,000-15,000 | IU/kg feed | 8-12,500 | IU/kg feed |
| Vitamin D3 | 1800-2000 | IU/kg feed | 3000-5000 | IU/kg feed |
| Vitamin E | 60-100 | mg/kg feed | 150-240 | mg/kg feed |
| Vitamin K3 | 2-4 | mg/kg feed | 2-4 | mg/kg feed |
| Vitamin B1 | 2-4 | mg/kg feed | 2-3 | mg/kg feed |
| Vitamin B2 | 6-10 | mg/kg feed | 7-9 | mg/kg feed |
| Vitamin B6 | 4-8 | mg/kg feed | 3-6 | mg/kg feed |
| Vitamin B12 | 0.03-0.05 | mg/kg feed | 0.015-0.04 | mg/kg feed |
| Niacin | 30-50 | mg/kg feed | 50-80 | mg/kg feed |
| (Vitamin B3) | | | | |
| Pantothenic acid | 20-40 | mg/kg feed | 10-18 | mg/kg feed |
| Folic acid | 1-2 | mg/kg feed | 1-2 | mg/kg feed |
| Biotin | 0.15-0.4 | mg/kg feed | 0.15-0.3 | mg/kg feed |
| Choline chloride | 200-400 | mg/kg feed | 300-600 | mg/kg feed |

Other Feed Ingredients

The composition of the invention may further comprise colouring agents, stabilisers, growth improving additives and aroma compounds/flavourings, polyunsaturated fatty acids (PUFAs); reactive oxygen generating species, anti-microbial peptides and anti-fungal polypeptides.

Examples of colouring agents are carotenoids such as beta-carotene, astaxanthin, and lutein.

Examples of aroma compounds/flavourings are creosol, anethol, deca-, undeca- and/or dodeca-lactones, ionones, irone, gingerol, piperidine, propylidene phatalide, butylidene phatalide, capsaicin and tannin.

Examples of antimicrobial peptides (AMP's) are CAP18, Leucocin A, Tritrpticin, Protegrin-1, Thanatin, Defensin, Lactoferrin, Lactoferricin, and Ovispirin such as Novispirin (Robert Lehrer, 2000), Plectasins, and Statins, including the compounds and polypeptides disclosed in WO 03/044049 and WO 03/048148, as well as variants or fragments of the above that retain antimicrobial activity.

Examples of antifungal polypeptides (AFP's) are the Aspergillus giganteus, and Aspergillus niger peptides, as well as variants and fragments thereof which retain antifungal activity, as disclosed in WO 94/01459 and WO 02/090384.

Examples of polyunsaturated fatty acids are C18, C20 and C22 polyunsaturated fatty acids, such as arachidonic acid, docosohexaenoic acid, eicosapentaenoic acid and gamma-linoleic acid.

Examples of reactive oxygen generating species are chemicals such as perborate, persulphate, or percarbonate; and enzymes such as an oxidase, an oxygenase or a syntethase.

The composition of the invention may further comprise at least one amino acid. Examples of amino acids which are used in animal feed are lysine, alanine, beta-alanine, threonine, methionine and tryptophan.

Uses

Use in Animal Feed

A GH30 glucuronoxylan hydrolase of the invention may also be used in animal feed. In an embodiment, the present invention provides a method for preparing an animal feed composition comprising adding one or more GH30 glucuronoxylan hydrolases of the present invention to one or more animal feed ingredients.

The one or more GH30 glucuronoxylan hydrolases of the present invention may for example be used to stabilize the healthy microflora of non-ruminant animals, in particular livestock such as, but not limited to pigs or swine (including, but not limited to, piglets, growing pigs, and sows), poultry (including, but not limited to, geese, turkeys, ducks and chicken such as broilers, chicks and layers); and rabbits but also in fish (including but not limited to salmon, trout, tilapia, catfish and carps; and crustaceans (including but not limited to shrimps and prawns)) by suppressing growth/intestinal colonization of viral (such as Coronaviridae, Porcine reproductive and respiratory syndrome virus (PRRSV), Persivirus coursing Bovin virus diarre and likewise), parasitic pathogens (coccidian protozoa, Eimeria maxima, Eimeria mitis) or bacterial pathogens such as Clostridium perfringens, Escherichia coli, Campylobacter coli, C. hyointestinalis and C. jejuni, Yersinia ssp., Treponema suis, Brachyspira hyodysenteriae, Lawsonia intracellularis and Salmonella, such as Salmonella enterica, Salmonella typhimurium and Salmonella mbandaka. In a preferred embodiment a GH30 glucuronoxylan hydrolase is applied to chicken and has anti-microbal activity against Clostridium perfringens. In a further embodiment a GH30 glucuronoxylan hydrolase of the present invention is used as a feed additive, where it may provide a positive effect on the microbial balance of the chicken digestive tract and in this way improve animal performance.

The one or more GH30 glucuronoxylan hydrolases of the present invention may also be used in animal feed as feed enhancing enzymes that improve feed digestibility to increase the efficiency of its utilization according to WO 00/21381 and WO 04/026334.

In a further embodiment a GH30 glucuronoxylan hydrolase of the present invention may be used as a feed additive, where it may provide a positive effect on the animals digestive tract and in this way improve animal performance in accordance to weight gain, feed conversion ratio (FCR), or improved animal health such as decreased mortality rate. FCR is calculated as the feed intake in g/animal relative to the weight gain in g/animal.

In the use according to the invention the GH30 glucuronoxylan hydrolases can be fed to the animal before, after, or simultaneously with the diet. The latter is preferred.

In a particular embodiment, the form of the GH30 glucuronoxylan hydrolase when it is added to the feed or when it is included in a feed additive is well-defined. Well-defined means that the GH30 glucuronoxylan hydrolase preparation is at least 50% pure as determined by Size-exclusion chromatography (see Example 12 of WO 01/58275). In other particular embodiments the GH30 glucuronoxylan hydrolase preparation is at least 60, 70, 80, 85, 88, 90, 92, 94, or at least 95% pure as determined by this method.

A well-defined GH30 glucuronoxylan hydrolase preparation is advantageous. For instance, it is much easier to dose correctly to the feed a GH30 glucuronoxylan hydrolase that is essentially free from interfering or contaminating other GH30 glucuronoxylan hydrolases. The term dose correctly refers in particular to the objective of obtaining consistent and constant results, and the capability of optimizing dosage based upon the desired effect.

For the use in animal feed, however, the GH30 glucuronoxylan hydrolase need not be pure; it may e.g. include other enzymes, in which case it could be termed a GH30 glucuronoxylan hydrolase preparation.

The GH30 glucuronoxylan hydrolase preparation can be (a) added directly to the feed, or (b) it can be used in the production of one or more intermediate compositions such as feed additives or premixes that is subsequently added to the feed (or used in a treatment process). The degree of purity described above refers to the purity of the original GH30 glucuronoxylan hydrolase preparation, whether used according to (a) or (b) above.

### EXAMPLES

### Example 1

Exogenous Glucuronoxylan Hydrolase from Glycoside Hydrolase Family 30 (GH30) Promotes Microbial Diversity and Butyrate Production in Cecal Broiler Fermentations of Maize Fibre In Vitro.

In this in vitro study, an endo-acting glucuronoxylan hydrolase from the glycoside hydrolase (GH) family 30 of SEQ ID NO 1 was tested for its ability to solubilise glucurono-arabinoxylan from maize fibre and the subsequent effect of the oligosaccharides generated on broiler cecal microbiota composition. The enzyme significantly decreased (P<0.01) the insoluble maize fraction. In order to investigate the impact of oligosaccharide size on fermentation patterns, oligosaccharides generated by the of SEQ ID NO 1 were separated and isolated by size exclusion chromatography. Fermentation of fraction pools with a molecular weight of 4-10 kDa showed higher butyrate concentration compared to pools containing oligosaccharides with a lower MW (1-4 kDa and 100-500 Da). During in vitro fermentation, butyrate concentrations were significantly higher (P<0.01) after 24 and 48 h with addition of the enzyme in situ. Addition of the glucuronoxylan hydrolase increased diversity and significantly reduced (P<0.01) the growth of Gram-negative Bacteroides xylanisolvens, while bacteria from the Lachnospiraceae family were increased significantly (P<0.01) which are critically important butyrate-producing bacteria.

Materials and Methods

Maize fibre substrate with a dry-matter (DM) content of 97.54% containing 364 g/kg crude fibre, 25.5 g/kg starch, 375 g/kg protein, 98 g/kg fat and 46 g/kg ash was obtained from de-starching and de-proteinization with Termamyl and Alcalase (Novozymes, Bagsvaerd, Denmark). See Ravn et al. (2017), for a detailed production procedure. The chemical composition of the maize is representative of maize used in the monogastric feed industry (Cowieson, 2005). The maize fibre had approximately 110.5 g/kg DM xylose.

Purified mono component enzyme: glucuronoxylan hydrolase (EC 3.2.1.136) from the GH30_8 family with of SEQ ID NO 1, of SEQ ID NO 2 , of SEQ ID NO 3 were obtained from B. subtilis and expressed in Bacillus (B.) licheniformis.

Enzymatic Digestion of Maize Fibre

Maize fibre (100 mg) substrate (n=4) was incubated with of SEQ ID NO 1 (10 ppm). The enzyme dosage of 10 ppm is corresponding to the recommended dosage for other carbohydrases in the feed additive market. The incubation lasted 4 h at 40° C. while stirring (500 rpm) in 4 mL 0.1 M sodium acetate (NaOAc) buffer pH 5.0 with 5 mM Ca^{2+.}

NSP Analysis

Analysis of soluble and insoluble neutral non-cellulosic polysaccharides (NCP) constituents (NSP constituents without cellulose) was performed on the maize fibre incubated with and without GH30 in triplicates according to Theander et al. (1995). Solubilisation with 12 M H₂SO₄ was not performed in order to avoid swelling of cellulose.

Separation of AXOS by Size Exclusion Chromatography (SEC)

Supernatants from enzymatic digestion of maize fibre (as described in section 2.3) were concentrated by speed vacuum evaporation before separation using a Superdex 75 column (26/60) (GE Healthcare Life Sciences, USA) at a flow rate of 150 mL/h in 50 mM ammonium formate (pH 5) equipped with a refractive index (RI) and Ultra violet (UV) detector. Fractions were collected at two-minute intervals. Pooled fractions: Pool I (fraction 22-30), Pool II (31-39), Pool III (40-53), Pool IV (55-59) and Pool V (60-70) were freeze dried, re-suspended in demineralized water and freeze dried again to remove ammonium formate.

In Vitro Cecal Fermentation

Maize fibre (150 mg) or 100 mg of Pool I-IV from SEC were diluted in anoxic sterile Moura medium prepared as described by Moura et al. (2007) modified according to De Maesschalck et al. (2015) and pH was adjusted to 6.5. Cecal content from 29-day-old broilers was mixed and diluted 10 times with Moura medium and from this dilution 150 μl was added to the maize fibre in a 15 ml final incubation volume to achieve a 1000 times dilution of cecal content. The fermentations were incubated for 48 h at 37° C., with or without glucuronoxylan hydrolase at an enzyme dosage of 10 ppm. Tubes with Pool I-IV did not receive enzyme supplementation. Fermentation supernatant was sampled after 24 and 48 h and stored at −20° C. until analysed. All fermentations were run in triplicates.

Short Chain Fatty Acid Analysis

The concentrations of SOFA in the cecal content of broilers and in the in vitro cecal fermentation supernatants were quantified by gas chromatography as described by Schäfer (1994). Samples were thawed and centrifuged before approximately 200 μl in vitro fermentation supernatant was mixed with 200 μl MeOH with 10% HCOOH. Lactate was quantified by HPLC (Dionex, Sunnyvale, USA) using a Rezex RoA column (Phenomenex, Torrance, USA) and a RI detector, after samples were diluted twice in 5 mM H₂SO₄ to achieve linearity on the RI detector.

DNA Extraction and PCR for Microbiota Composition

Total genomic bacterial DNA was extracted from in vitro cecal inoculum fermentation using a Nucleospin 96 soil kit (Macherey-Nagel, Germany) and a Genie-T vortexer (Scientific Industries Inc., USA). DNA was quantified with a fluorimetric Qubit dsDNA HS assay kit (Invitrogen, USA) and 10-15 ng extracted DNA was used in a PCR reaction (25 μl) targeting the V3-V4 variable regions of the 16S rRNA gene. 10-15 ng of extracted DNA was used as a PCR reaction template (25 μl) containing dNTPs (400 nM of each), Phusion® Hot Start II DNA polymerase HF (2 mU), 1× Phusion® High Fidelity buffer (New England Biolabs Inc., USA) and barcoded library adaptors (400 nM) containing V3-4 specific primers: forward primer (341F) CCTACGGGNGGCWGCAG and reverse primer (805R): GACTACHVGGGTATCTAATCC. The PCR settings used were: initial denaturation at 98° C. for 2 min, 30 cycles of 98° C. for 30 s, 52° C. for 30 s, 72° C. for 30 s and final elongation at 72° C. for 5 min.

16S rRNA Gene Sequencing of Cecal Microbiota Composition

The amplicon libraries were purified using the Agencourt® AMpure XP bead protocol (Beckmann Coulter, USA). The purified sequencing libraries were pooled and samples were paired end sequenced (280 bp×260 bp reads with dual indexes of 8 bp) on a MiSeq (Illumina, San Diego, Calif.) using a MiSeq Reagent kit v3, 600 cycles (Illumina) following the standard guidelines for preparing and loading samples on the MiSeq. Genomic DNA was spiked to overcome low complexity issues often observed with amplicon samples. Bioinformatics was performed as previously described by Ravn et al. (2017).

Bioinformatics Processing, OTU Clustering and Classification

The generation of OTU tables was done with usearch version 10.0.240 (Edgar, 2016). Primer binding regions were removed with fastx_truncate and reads were filtered to contain less than one error per read. The quality filtered reads were denoised with unoise3. OTU abundance was calculated by mapping with usearch_global using a 97% identity threshold. Taxonomical classification was done with the RDP classifier version 2.12.

Statistical Analysis

Analysis of variance was performed using the ANOVA procedure in the statistical package SAS JMP 12.1.0 (SAS Institute Inc., 2015). For NSP and SOFA concentrations in vitro, the effect of time of incubation, treatment and their interaction were included in the model. For significant models (P<0.05) least squared means were separated using the Tukey-Kramer HSD test (P<0.05) as provided in the ANOVA model. For soluble xylose, NSP data or SOFA concentration, following in vitro fermentation, the main effect of treatment was tested and comparisons made using the Tukey-Kramer test. Statistical analysis of 16S rRNA metagenomics data was handled in R using ANOVA (Ravn et al., 2017).

The microbiome data were analysed in R using the ampvis package v.1.9.1 (Albertsen et al., 2015), which builds on the R package DESeq2 (Love et al., 2014) for detecting species in differential abundance. An OTU was considered significantly differentially abundant if the adjusted p-value was below 0.05.

For the beta diversity analysis, the dissimilarity indices were calculated using the vegdist function from the vegan package (Oksanen et al., 2015) with the bray method. Permutational multivariate analysis of variance was analysed with adonis from the vegan package.

Results

NSP analysis

The solubilising effects of the GH30 on maize fibre NSP components were analysed for both the soluble (DP>10) and insoluble sugar fractions (Table 1). The enzyme significantly increased (P<0.05) the soluble arabinoxylan fraction (measured as arabinose and xylose following acid hydrolysis of the soluble fraction). Arabinoxylan solubilisation was also reflected by a significant reduction (P<0.05) in insoluble NSP. Likewise, solubilisation of rhamnogalacturonan structures (measured as rhamnose and galactose following acid hydrolysis of the soluble fraction) were also significantly increased (P<0.05).

TABLE 1
Average individual NSP content (g/kg DM) in the soluble and insoluble
fractions, their total content as well as after incubation with GH30 on
maize fibre (g/kg DM) (n = 3)

| Non-Starch | Treatments | | |
|---|---|---|---|
| Polysaccharides | Blank | GH30 | Pooled SEM¹ |
| Soluble | | | |
| Rhamnose | 0.1^{b} | 0.5^{a} | 0.07 |
| Fucose | 0.0^{b} | 0.0^{ab} | 0.01 |
| Arabinose | 1.2^{b} | 27.9^{a} | 1.14 |
| Xylose | 1.0^{b} | 25.2^{a} | 1.17 |
| Mannose | 0.1^{a} | 0.1^{a} | 0.07 |
| Galactose | 0.3^{b} | 2.1^{a} | 1.49 |
| Glucose | 0.4^{a} | 0.9^{a} | 0.15 |
| Sum of soluble NSP | 3.0 | 56.8 | — |
| Ratio: Arabinose/Xylose | 1.16^{a} | 1.11^{ab} | 0.05 |
| Insoluble | | | |
| Rhamnose | 2.8^{a} | 2.2^{b} | 0.10 |
| Fucose | 0.3^{a} | 0.4^{a} | 0.10 |
| Arabinose | 105.0^{a} | 59.2^{b} | 2.30 |
| Xylose | 109.5^{a} | 67.1^{b} | 2.0 |
| Mannose | 14.8^{a} | 14.6^{a} | 0.30 |
| Galactose | 15.2^{a} | 12.6^{b} | 0.50 |
| Glucose | 133.4^{a} | 133.4^{a} | 9.00 |
| Sum of insoluble NSP | 380.8 | 289.4 | — |
| Ratio: Arabinose/Xylose | 0.96^{a} | 0.88^{b} | 0.01 |
| Total NSP | 383.8 | 346.2 | — |

Table 2 shows the in vitro SOFA production (mM) from 24 h and 48 h cecal fermentations of maize fibre at 37° C. using cecal broiler content as inoculum incubated without or with GH30 dosed at 10 ppm (n=3) as performed in Example 1. In vitro cecal fermentation of maize fibre supplemented with GH30 significantly increased (P<0.05) certain SOFA, in particular butyrate (table 2). The GH30 treatment showed an increase in butyrate formation of 3.8 mM higher than control, due to a higher total GAX solubilisation degree by the GH30 as seen in Table 1.

| Time point 24 h | | | 48 h | | Pooled |
|---|---|---|---|---|---|
| Group | Blank | GH30 | Blank | GH30 | SEM¹ |
| Acetate | 20.6^{b} | 41.2^{a} | 89.0^{b} | 107.6^{a} | 23.77 |
| Propionate | 8.1^{a} | 7.2^{a} | 15.0^{a} | 16.8^{a} | 5.78 |
| lsobutyrate | 1.4^{a} | 0.9^{a} | 3.8^{a} | 2.5^{b} | 0.41 |
| Butyrate | 3.2^{b} | 4.9^{a} | 5.3^{b} | 9.1^{a} | 0.96 |
| lsovalerate | 2.9^{a} | 2.1^{a} | 6.6^{a} | 4.5^{b} | 0.75 |
| Valerate | 0.6^{a} | 0.2^{b} | 11.2^{a} | 5.3^{b} | 1.10 |
| Total SCFA | 36.6^{a} | 56.4^{a} | 130.9^{b} | 145.7^{a} | 25.22 |

¹SEM = Standard error of mean summation of both 24 h and 48 h.
^{abc}Mean values within a column not sharing a common letter differ significantly (P < 0.05; Tukey-Kramer HSD) (n = 3). The two timepoints were tested

SEC of Oligomers Present in Supernatant of Maize Fibre Incubated with GH30

Pools I (22-30), II (31-39), III (40-53), IV (55-59) and V (60-70) were fractionated by SEC as shown in FIG. 1. The average sizes of the fractions correspond approximately to: Pool I: 10-30 kDa; Pool II: 4-10 kDa; Pool III: 1-4 kDa and Pool IV: 100-500 Da. Pool V: <100 Da. High UV signal (280 nm) peaks matches the RI signal indicating oligomers containing light absorbance compounds.

In Vitro Fermentation

Fermentation of maize fibre supplemented or not with GH30 in combination with inoculum from pooled cecal content from two 29-day-old broilers was performed to investigate SOFA production. The GH30 significantly increased butyrate levels (P<0.05) after 24 h and 48 h and total SOFA after 48 h (P<0.05). Addition of GH30 also significantly decreased (P<0.05) branched SOFA compared to control. No detectable lactate was present in the samples.

Fermentation of isolated AXOS fractions (Pool I-IV) in combination with inoculum from pooled cecal content from two 29-day-old broilers was performed to investigate SOFA production affected by AXOS size. After 24 h and 48 h, Pool I-Ill significantly increased (P<0.05) total SOFA production compared to Pool IV. Pool II yielded significantly higher (P<0.05) butyrate at 24 h and numerically higher butyrate after 48 h compared to the other pools. Pool V was not included in the fermentations due to too little material mass.

Microbiota Composition Analysis

Except for the analysis of differential abundance with Deseq2, samples were rarefied prior to analysis.

Analysis of Alpha Diversity

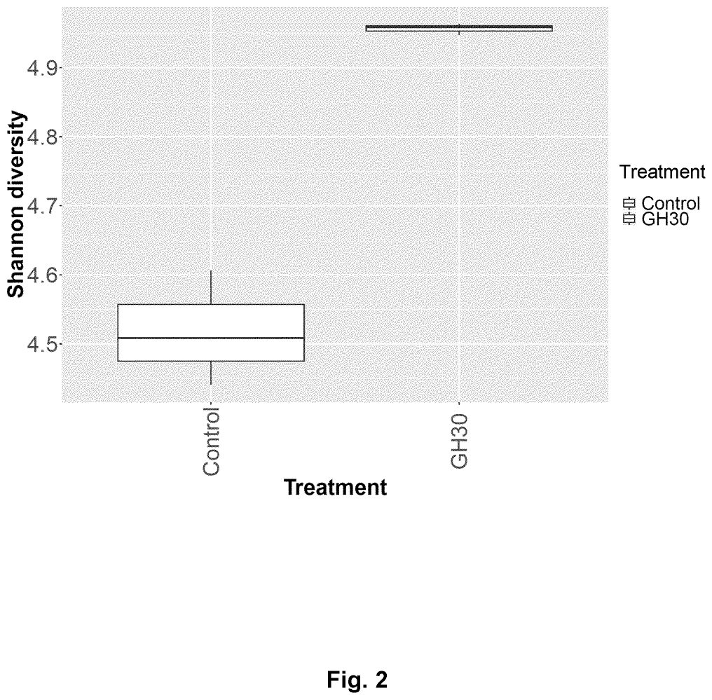

The effect of treatment on alpha diversity (shannon index) was analysed with ANOVA. The analysis showed that the shannon index was significantly associated with enzymatic treatment (p-value<0.001). A boxplot of the shannon index is shown in FIG. 2.

Analysis of Beta Diversity

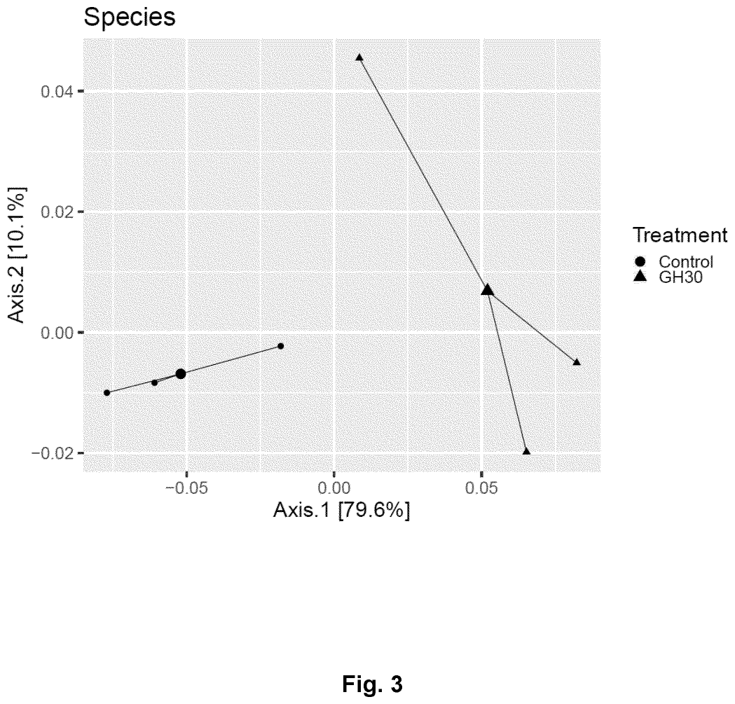

Beta diversity was analysed using the weighted Unifrac index. The effect of treatment on beta diversity was investigated with permutational manova (adonis from the vegan package). Beta diversity was found to be non-significantly associated with enzymatic treatment (R2 0.63 p-value 0.1). The association between beta diversity and treatment is visualised in FIG. 3.

Differential Abundance

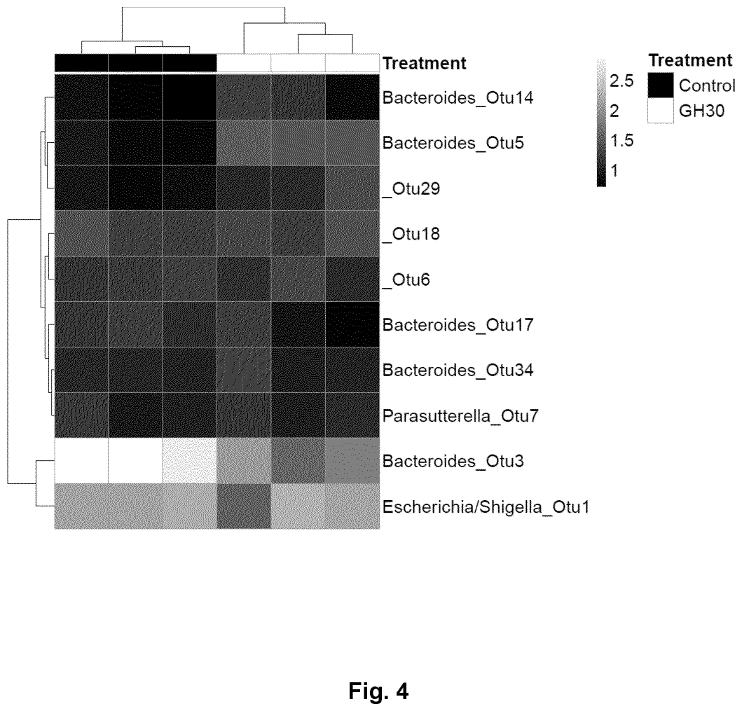

The top 20 most abundant species were clustered with the hclust function from the R package. The OTU counts were log transformed before the clustering. The clustering is visualized with a heatmap in FIG. 4. The heatmap shows that samples cluster according to treatment. The heatmap also shows that there is a decrease in one Bacteroides species (OTU3) and an increase in another Bacteroides species (OTU5) upon enzymatic treatment with GH30.

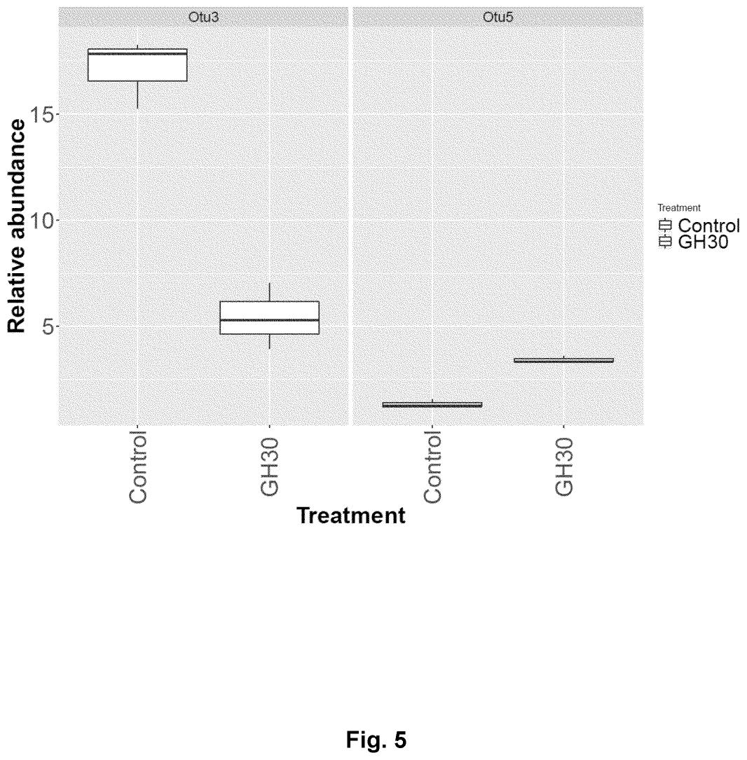

Differential abundance analysis with Deseq2 revealed that the Bacteroides species (OTU3) was significantly decreased in all groups treated with xylanase. The abundances of the three OTU's are shown in FIG. 5.

Sequence analysis of the two OTU's revealed that OTU3 is 99% identical to the 16S rRNA sequence of Bacteroides xylanisolvens and that OTU5 is 100% identical to Bacteroides dorei/vulgatus.

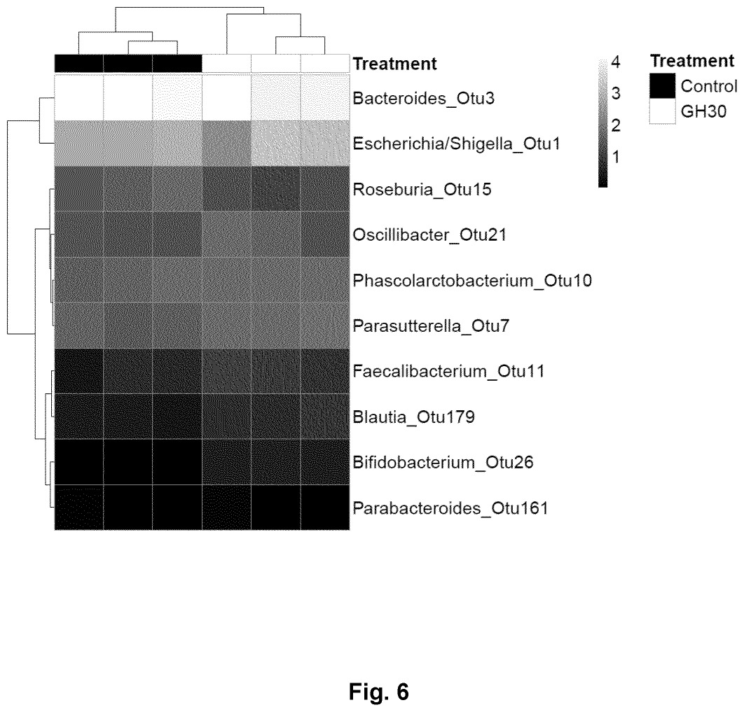

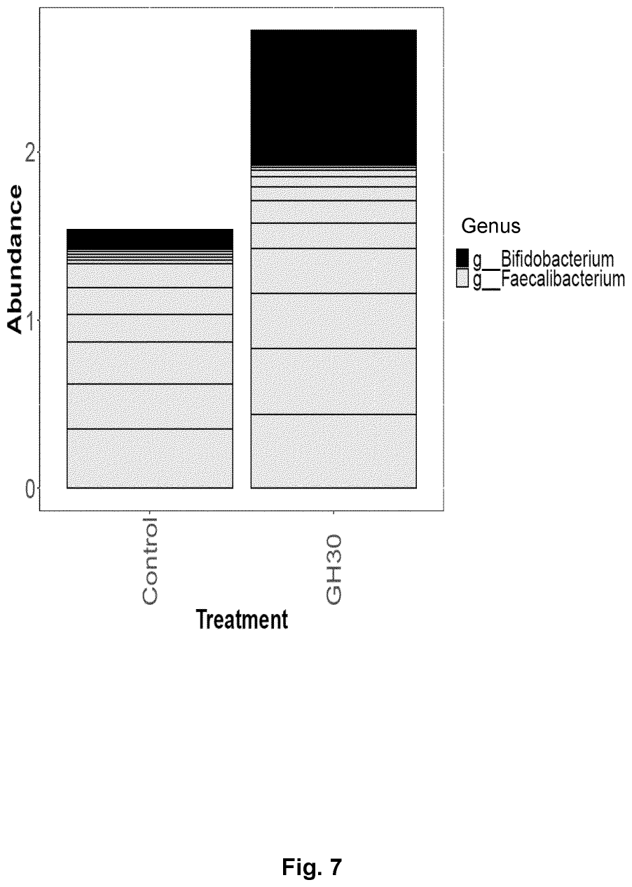

A hierarchical clustering of the 10 most abundant genera is shown in FIG. 6. The abundance of the genera was obtained by merging all OTU's belonging to the same genus. The heatmap show an increase in genus Faecalibacterium and genus Bifidobacterium. Analysis with deseq2 revealed that the increase in Bifidobacterium was significant (P<0.001). The relative abundance of Faecalibacterium and Bifidobacterium are shown in FIG. 7.

Microbiome Analysis of Fractions (I-IV)

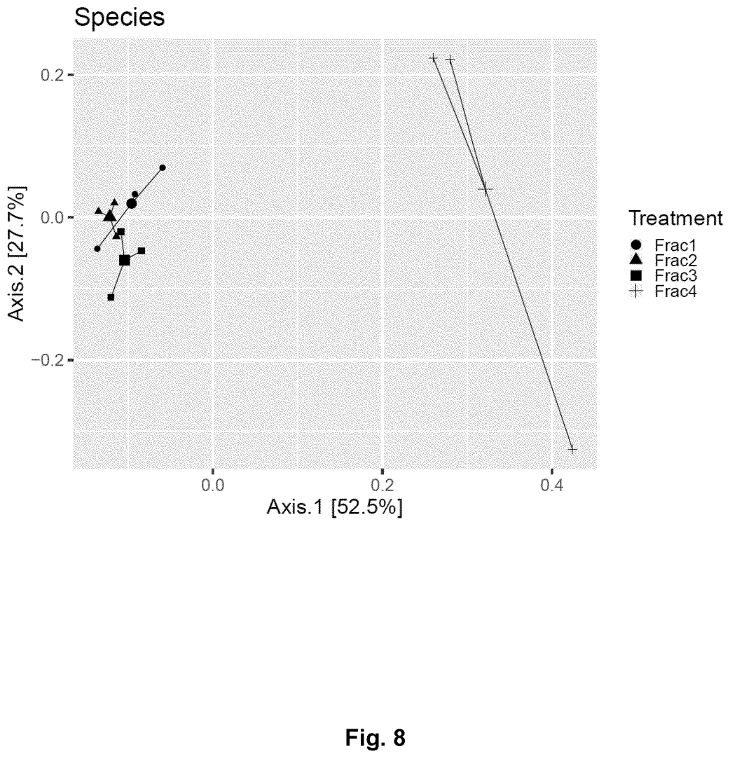

No significant effect on alpha diversity was found between fraction I-IV from the size exclusion of the GH30 supernatant. Beta diversity was analysed using the weighted Unifrac index. The effect of fractions on beta diversity was investigated with permutational manova (adonis from the vegan package). Beta diversity was found to be significantly associated between fractions I-III and IV (R2 0.6 p-value 0.001). The association between beta diversity and treatment is visualised in FIG. 8.

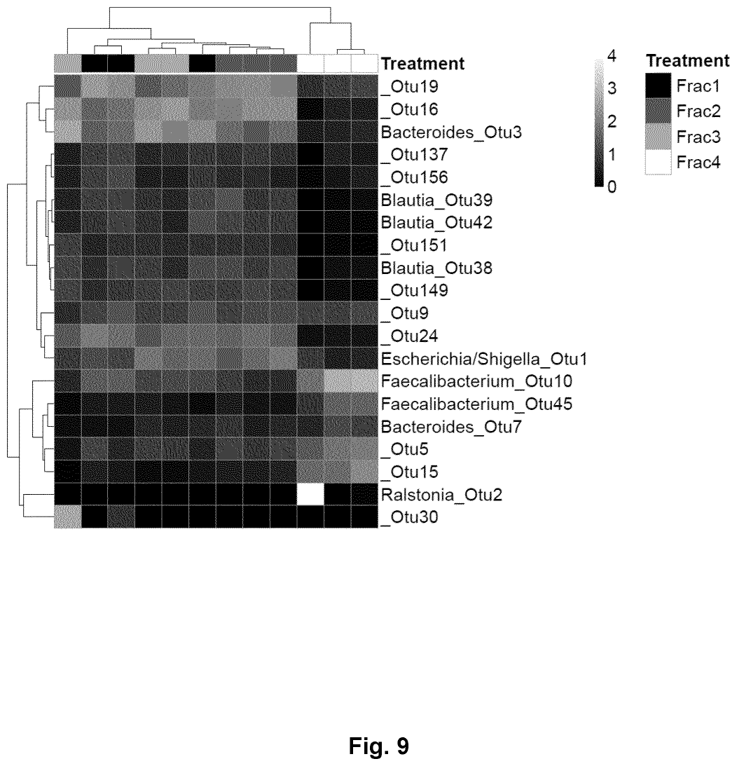

The top 20 most abundant species were clustered with the hclust function. The OTU counts were log transformed before the clustering. The clustering is visualized with a heatmap in FIG. 9. The heatmap shows that the samples from fraction IV cluster together.

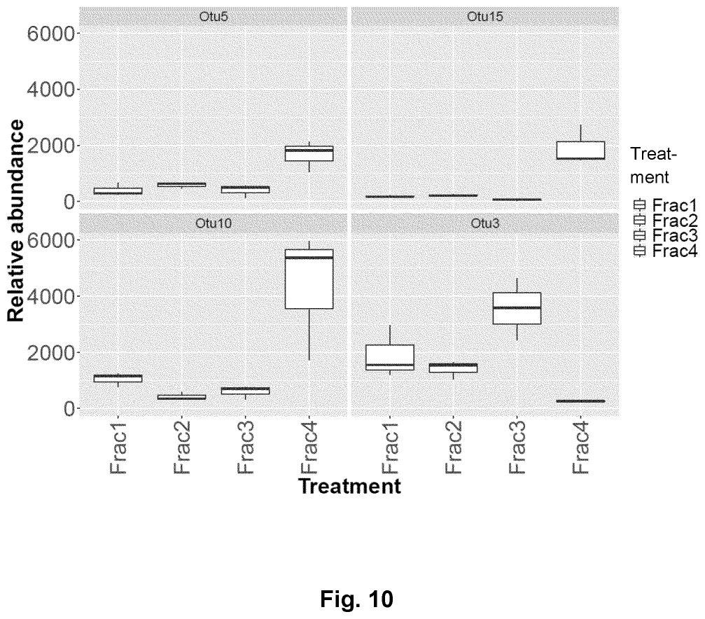

FIG. 10 shows that a decrease in one Bacteroides species (OTU3) while there is an increase in other Bacteroides species (OTU5, OUT15 and OTU10) with fraction IV supplementation.

### Example 2

The GAX-specific glucuronoxylan hydrolase improved performance and morphological gut health parameters in broilers fed a maize/DDGS/soy diet.

Materials and Methods

Chickens were housed and euthanized in accordance Belgian animal welfare regulations (2010/63/EC).

Animals and Diets

A total of 480 newly hatched male Ross-308 broiler chicks were randomly divided into 16 pens with 30 chicks per pen, (8 pens per treatment group) and housed on solid floors covered with wood shavings. The light schedule provided a 18 h light/6 h darkness cycle with 23 h light/1 h darkness cycle during the first 4 days of the trial. The room temperature of the stable was adapted and optimized according to the bird's' requirements by a central heating system. All birds were fed the same maize-soy-based mash feed diet either as supplemented or un-supplemented with of SEQ ID NO 1. The diets (table 1) were formulated in order to meet energy and digestible amino acids requirements for the broilers in the two phases (starter and grower, 0-7, and 7-29 d, respectively).

Treatments

Purified mono component glucuronoxylan hydrolase (EC 3.2.1.136) class GH30_8 of SEQ ID NO 1 was obtained from B. subtilis and expressed in B. licheniformis). The dosage of the enzyme used was 10 ppm. The enzyme was added to the diets in liquid form by spraying 1.5 L diluted enzyme-solution onto 30 kg of ground maize premix. The premixes were stored in plastic bags at 4° C. until finally mixed into the mash feed rations to achieve a final concentration of 2 ml/kg feed corresponding to a 10 ppm/kg feed enzyme concentration.

Sample and Data Collection

Feed intake and body weight were obtained on a pen level at day 7, 14, 21, and 29. At day 14 and at 29, five chickens per pen were euthanised and weighed individually. Jejunum digesta from proximal jejunum to Meckel's diverticulum and cecal digesta (both ceca) were collected and snap-frozen in liquid nitrogen. Epithelium from the duodenum loop was obtained and fixed in 4% formaldehyde solution (BiopSafe ® containers, Ax-lab, Denmark, http://www.axlab.dk/).

Intestinal Dysbacteriosis and Inflammation Scoring of Birds

Immediately after euthanasia an experienced veterinarian scored each bird macroscopically. Each chicken was given a score between 0-10 for intestinal dysbacteriosis parameters. 0 being normal gastrointestinal tract and 10 being the most severe dysbacteriosis (Teirlynck et al. 2011). In summary, parameters scored were (1) over all gut ballooning; cranial and caudal (2) inflammation/significant redness of the serosal/mucosal side of the gut; (3) fragility of the gut wall; (4) flaccidity/thickness of the gut wall; (5) abnormal content; (6) undigested feed. See a detailed description by Teirlynck et al. (2011).

In Vitro Cecal Fermentations

For in vitro fermentation, maize fibres were obtained by using a procedure that includes two incubations with an alpha-amylase (Termamyl® Novozymes, Denmark) and an incubation with a protease (Alkalase®, Novozymes, Denmark). Analysis of the fibres showed that the material contains 36.4% crude fibre, 2.55% starch, 37.5% protein, 9.8% fat and 4.6% ash approximately 110.5 g/kg xylose. A slurry of maize fibre (1% w/v) diluted in anoxic sterile Moura medium (low-nutrient medium) was prepared as described by Moura et al. (2007) and modified according to De Maesschalck et al. (2015). Fermentation pH was adjusted to 6.5 before placing the samples in an anaerobic cabinet. Cecal content from the 29-day old broilers was mixed and diluted 10 times with Moura medium and from this dilution 150 pl was added to the maize fibre in a 15 mL final incubation volume to achieve a 1000x dilution of cecal content. The fermentations were incubated for 48 h at 37° C., with or without of SEQ ID NO 1, 2, or 3 at an enzyme dosage of 10 ppm. Fermentation supernatant was sampled after 6, 24 and 48 h and stored at −20° C. until analysed. All fermentations were done in biological triplicates and the fermentation experiment was repeated twice on separate days.

Analytical Procedures

The concentration of SOFA in the cecal content of broilers and in the in vitro cecal fermentation supernatants were quantified by gas chromatography as described by Schäfer (1994). Samples were thawed and centrifuged before approximately 100 mg cecal content or 200 μl in vitro fermentation supernatant was mixed with 200 μl MeOH with 10% HCOOH. Lactate was quantified by HPLC (Dionex, USA) using a Rezex RoA column (Phenomenex, Denmark) and a RI detector, after samples were diluted in 5 mM H₂SO₄ to achieve linearity on the RI detector.

Jejunum digesta samples were divided in two equal portions, one for freeze drying and one for liquid fractionation, following defrosting and pooling by pen with 5 birds per pen per sampling point. The liquid fractionation was performed by centrifugation (15 min, 4000 rpm), at room temperature, followed by harvesting of the supernatant.

The nitrogen content was measured by Dumas method (FP628 Nitrogen analyzer, LECO corporation, USA); and crude protein in feed rations was determined from correcting the nitrogen concentration by 6.25.

The diets and pooled freeze-dried jejunum digesta from three pens of each treatment were analysed for soluble and insoluble NSP according to Theander et al. (1995). Each treatment was done in triplicate (400 mg per treatment was analyzed).

The viscosity of the liquid fraction (supernatant) of the jejunal content was measured in a ViPr (viscosity pressure) assay (Abel & Pettersson, WO2011107472 A, 2011). Supernatants (300 μl) were transferred to a 96 well microtiter plate (Nunc™, Denmark) and viscosity measurements (n=3) were performed using a Hamilton Microlet® Starlet liquid handler (Hamilton company, USA). The liquid handle measures viscosity by the pressure differences detected in the Hamilton pipettes.

Confocal Microscopy and Immunocytochemistry

Confocal laser scanning microscopy (CLSM) was performed on immunolabelled jejunum digesta samples as described in Ravn et al. (2016) using a CLSM SP2 microscope (Leica, Heidelberg, Germany). A 63× water-immersion objective was used for all images. Images were processed in the LAS AF Lite (Leica) software and the signals of the 543-nm laser and the 488-nm laser were coloured in red and a green, respectively.

Immunolabelling of the freeze-dried jejunum digesta was performed as follows: Approx. 100 mg of freeze dried jejunum digesta was mixed with melted 2% agar and left to solidify at room temperature. Small square pieces of agar-embedded digesta were fixated, dehydrated, embedded in paraffin and sectioned (Ravn et al., 2016). Deparaffinated thin sections (4 μm) of digesta were labelled with antibodies by immunocytochemistry techniques (Ravn et al., 2016). Sections were blocked with skimmed milk and washed in PBS buffer and incubated with 10-fold dilutions of skimmed milk in 1× PBS for 1 hour with LM27 and LM28 rat primary monoclonal antibody that specifically binds substituted AX regions (Cornuault et al., 2015). Samples were subsequently incubated in a 300-fold dilution of anti-rat IgG linked to an Alexa-555 fluorophore for 1½ h and washed in PBS buffer. Citiflour AF1 (Agar Scientific, UK) anti-fading agent was added to avoid bleaching of fluorescent signals.

Morphological Examination

Duodenum segments taken at the duodenum loop were fixed in 4% formaldehyde solution (BiopSafe®, Ax-lab, Denmark). Samples were dehydrated in xylene and a series of graded ethanol, embedded in paraffin and sectioned in 4 μm sections. Samples were deparaffinated, stained with hematoxylin and eosin and examined using a digital light microscope DM LB2 (Leica, Heidelberg, Germany) with a DFC 320 camera (Leica, Heidelberg, Germany). The villus length of all samples was measured by random measurement of 10 villi per duodenum section using the image analysis system LAS v4.0 (Leica Application suite V4).

Immunohistochemical Examination of CD3 T-Cells

Deparaffinated sections of the duodenum (3 samples per pen with a total number of 24 per treatment) were prepared for immunolabeling with a pressure cooker antigen retrieval method (Tender Cooker; Nordic Ware, Minneapolis, Minn., USA) using 10 mM citrate buffer, pH 6. Immunohistochemical labelling of leucocytes was performed with antibodies specific for CD3 positive T-cells with Dako CD3 (A0452) (Dako, Glostrup, Denmark). Sections were washed in Dako Autostainer+ washing buffer and blocked with peroxidase reagent for 5 mins and rinsed with Dako washing buffer. Sections were incubated with primary antibody for 30 min at room temperature and diluted 100× in Dako antibody diluent (S3022). Sections were rinsed again in Dako washing buffer and incubated with labelled polymer-HRP (DAB) (K4011) for 30 min at room temperature. Sections were then washed twice with Dako washing buffer and Dako DAB+ substrate and DAB+ chromogen was added for 5 mins. The staining was stopped and counterstained with haematoxylin for 10 mins and washed for 1 min under running water. Sections were dehydrated with xylene and a graded series of ethanol, and mounted. Brown-stained leucocytes were quantified by area % using a colour threshold application in the image analysis system LAS v4.0 software (Leica).

DNA Extraction and PCR for Microbiota Composition and qPCR Analysis

Total genomic bacterial DNA was extracted from cecal material using a Nucleospin 96 soil kit (Macherey-Nagel, Germany) and a Genie-T vortexer (Scientific Industries Inc., USA). DNA was quantified with a fluorimetric Qubit dsDNA HS assay kit (Invitrogen, USA) and 10-15 ng extracted DNA was used in a PCR reaction (25 μl) targeting the V3-V4 variable regions of the 16S rRNA gene. 10-15 ng of extracted DNA was used as a PCR reaction template (25 μl) containing dNTPs (400 nM of each), Phusion® Hot Start II DNA polymerase HF (2 mU), 1× Phusion® High Fidelity buffer (New England Biolabs Inc., USA) and barcoded library adaptors (400 nM) containing V3-4 specific primers: Forward primer (341F) CCTACGGGNGGCWGCAG and reverse primer (805R): GACTACHVGGGTATCTAATCC. The PCR settings used were: Initial denaturation at 98° C. for 2 min, 30 cycles of 98° C. for 30 s, 52° C. for 30 s, 72° C. for 30 s and final elongation at 72° C. for 5 min.

qPCR on Total Bacteria and Butyryl-CoA:Acetate-CoA Transferase Gene

Total number of bacteria and butyrate-CoA:acetate CoA transferase gene were quantified in three chickens per pen (16 samples per treatment). Purified cecal DNA was diluted ×100 to match the standard curves. The total number of bacteria was determined using the following primers Fwd: (5′-CGGYCCAGACTCCTACGGG-3′) and Rev: (5′-TTACCGCG GCTGCTGGCA-3′) (Hopkins et al., 2005). To quantify the number of gene copies encoding the butyryl-CoA: acetate-CoA transferase enzyme, primers fwd: (5′-GCIGAICATTTCACITGGAAYWSITGGCAYATG-3′) and rev: (5′-CCTGCCTTTGCAATRTCIACRAANGC-3′) were used (Louis and Flint 2007). Each reaction was done in triplicate in a 12 μl total reaction mixture using 2× SensiMix SYBR No-ROX mix. Final primer concentrations and PCR cycles were prepared as described by De Maesschalck et al (2015). Amplification and fluorophore detection were performed using a CFX384 Bio-Rad detection system (Bio-Rad, Nazareth-Eke, Belgium).

16S rRNA Gene Sequencing of Cecal Microbiota Composition

The amplicon libraries were purified using the Agencourt® AMpure XP bead protocol (Beckmann Coulter, USA). The purified sequencing libraries were pooled and samples were paired end sequenced (280 bp×260 bp reads with dual indexes of 8 bp) on a MiSeq (Illumina, San Diego, Calif.) using a MiSeq Reagent kit v3, 600 cycles (Illumina) following the standard guidelines for preparing and loading samples on the MiSeq. Genomic DNA was spiked to overcome low complexity issues often observed with amplicon samples. Bioinformatics was performed as previously described by Ravn et al. (2017).

Statistical Analysis

Growth performance, digesta, morphology and in vitro fermentation data were subjected to statistical analyses using ANOVA multiple comparison with the statistical package SAS JMP. For comparison of pairs of more than two means the Tukey-Kramer HSD model was used with individual pens as determining factor. For villus length, the average of 10 villi from one bird was used as one data value. Statistical analysis of 16S rRNA metagenomics data was handled in R using ANOVA (Ravn et al., 2017).

Results

Bird Performance

Feed intake was not significantly affected by the supplementation with SEQ ID NO 1 (table 2). Total mortality was 5.0% (12 birds) for the control group and 5.4% (13 birds) for the GH30 treatment group during the whole trial period.

Body weight (BW) and live weight gain (LWG) were increased significantly (P<0.001) with GH30 supplementation after 29 days (table 2). Feed conversion ratio (FCR) was in general lowered with the GH30 supplementation (P<0.001, table 2).

TABLE 2
Effect of enzyme supplementation on growth performance of birds.

| | (day) | | | | | | | | |
|---|---|---|---|---|---|---|---|---|---|
| | 8 | | 14 | | 21 | | 29 | | |
| | | SEQ | | SEQ | | SEQ | | SEQ | |
| | | ID | | ID | | ID | | ID | Pooled |
| Treatment | Control | NO 1 | Control | NO 1 | Control | NO 1 | Control | NO 1 | SEM¹ |
| FCR | 1.13 | 1.12 | 1.42 | 1.30 | 1.47 | 1.36 | 1.55 | 1.43 | 0.10 |
| P value² | | 0.86 | | <0.01 | | <0.01 | | <0.01 | |
| FI (g/day) | 21.4 | 21.0 | 54.6 | 52.4 | 91.1 | 90.0 | 133.5 | 138.7 | 7.76 |
| P value² | | 0.36 | | 0.08 | | 0.79 | | 0.10 | |
| BW (g) | 191.0 | 189.3 | 421.3 | 430.8 | 854.1 | 893.75 | 1545.8 | 1672.1 | 56.86 |
| P value² | | 0.76 | | 0.21 | | <0.01 | | <0.01 | |
| LWG | 19.0 | 18.8 | 38.4 | 40.3 | 62.1 | 66.3 | 86.5 | 97.33 | 6.00 |
| (g/day) | | | | | | | | | |
| P value² | | 0.67 | | 0.03* | | <0.01 | | <0.01 | |

¹SEM = standard error of mean.
Feed conversion ratio (FCR), feed intake (FI), body weight (BW) and live weight gain (LWG) were calculated at four time intervals.
Values are the means for 8 pens with 30 chickens (25 and 20 chickens after day 14 and 29, respectively).
²P-value: Pairwise comparison of means (Tukey-Kramer HSD test) for control and enzyme supplementation up to days 8, 14, 21 and 29, respectively.

NSP of Jejunum Digesta from 29-Day-Old Broilers

Insoluble NSPs were decreased considerably (P<0.05) in jejunum digesta of birds supplemented with GH30 enzymes of the invention(SEQ ID NO 1). The GH30 enzyme lowered total insoluble GAX compared to control as seen in table 3. Likewise, soluble NSPs were increased (P<0.05). Interestingly, small amounts of soluble oligomers containing rhamnose (0.1 g/kg DM), mannose (0.45 g/kg) and galactose (3.5 g/kg) also increased (P<0.05) with GH30 supplementation.

Viscosity of Jejunum Digesta

No significant effect on enzyme supplementation on average jejunum digesta viscosity was observed (control=698.31 Pa and SEQ ID NO 1=704.88 Pa).

Microscopy (GAX Solubilisation)

A strong signal from maize GAX was seen in control jejunum digesta samples using both antibodies LM27 and LM28. Both antibody signals were decreased or completely disappeared in the digesta of chickens supplemented with SEQ ID NO 1.

Dysbacteriosis/Inflammation Scoring of the Intestine

Supplementation with GXH did not significantly affect macroscopic dysbacteriosis scoring, as described by Teirlynck et al. (2011). The average macroscopic dysbacteriosis score at day 14 was 1.55 and 2.0 (out of 10) for control and GH30-supplemented birds, respectively. At day 29 the average macroscopic dysbacteriosis score was 4.73 and 4.55 for control and SEQ ID NO 1-supplemented birds, respectively.

Intestinal Morphology

GH30 addition to the diet was associated with a significantly increased villi length (P<0.001) in the duodenum (Table 4). Compared to control, birds of 14 and 29 days of age supplemented with the GH30 had an increase in villi length of 292.4 μm and 302.4 μm, respectively.

TABLE 4
Villus length in the duodenum affected by
xylanase supplementation

| | Day 14 | | | Day 29 | | | |
|---|---|---|---|---|---|---|---|
| | Villi | | P | Villi | | P | Pooled |
| Treatment | (μm) | length | value² | (μm) | length | value² | SEM¹ |
| Control | 1644 | | — | 1918 | | — | 56.6 |
| (n = 24) | | | | | | | |
| GXH (n = 24) | 1936 | | <0.01 | 2221 | | <0.01 | 66.1 |

¹SEM = Standard error of mean. Random measurements of 10 villi per duodenum sections of every sampled chicken was performed by a computer-based analysis system.
²P value. Comparison of means for control and enzyme supplementation at days 14 and 29 (Tukey-Kramer HSD test).

Furthermore, the area % of CD3 T-cell infiltration was significantly lower (P<0.001) (Table 5), indicating a lower inflammation level in birds supplemented with xylanase.

TABLE 5
Effect of xylanase supplementation on CD3 T-cell infiltration in
the duodenum as quantified by area %²

| | Day14 | | Day29 | | |
|---|---|---|---|---|---|
| | CD3 T-cell | P | CD3 T-cell | P | Pooled |
| Treatment | (area %²) | value³ | (area %²) | value³ | SEM¹ |
| Control | 8.4 | — | 11.80 | — | 1.21 |
| (n = 24) | | | | | |
| GXH (n = 24) | 5.0 | <0.01 | 6.68 | <0.01 | 0.94 |

¹SEM = Standard error of mean.
²The area % of brown stained CD3 T-cells in villi tissue was quantified using a color threshold tool in the LAS v.4 software (Leica). A Tukey-Kramer HSD test was done to compare all means of pairs between groups receiving non-supplemented and enzyme-supplemented diets (n = 24).
³P value. Comparison of means for control and enzyme supplementation at days 14 and 29 (Tukey-Kramer HSD test).

16S rRNA Analysis of Microbiota Composition

No significant difference in relative abundance (%) of genera was observed between control groups and SEQ ID NO 1 supplemented groups (n=40). However, a numerical increase was observed in certain Lachnospiraceae families with GH30 xylanase supplementation, which are known butyrate-producing bacteria (Hippe et al., 2011).

qPCR Analysis of Butyrate-CoA:Acetate CoA Transferase Gene

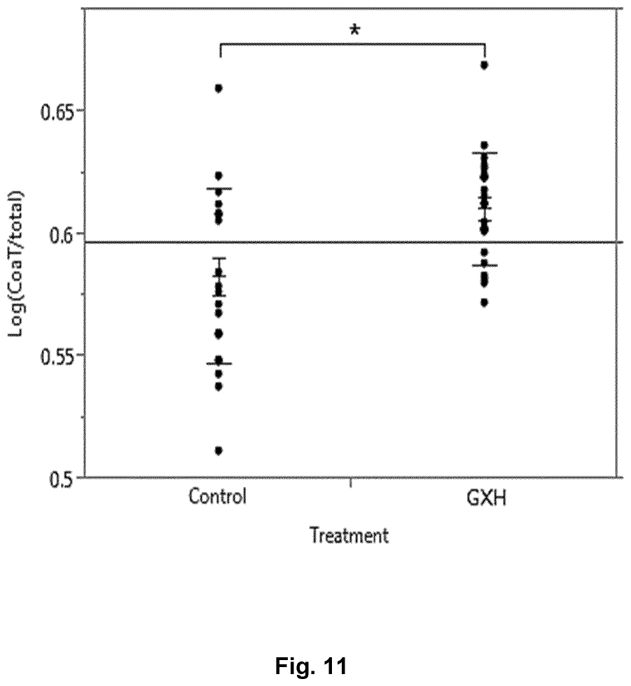

A significantly higher (P<0.0038) ratio between the butyryl-CoA:acetate CoA transferase gene and total amount of bacteria was observed with GH30 xylanase supplementation to the diet in 29-day-old broilers (FIG. 11).

SCFA Concentrations in Cecal Content

SCFA analysis of the cecal content showed a significant increase (P<0.05) in butyrate (+3.57 μmol/g) in broilers supplemented with SEQ ID NO 1 (Table 6).

TABLE 6
SCFA concentrations (μmol/g) in cecal content of broilers
supplemented without and with dietary GXH (n = 24)
(SEQ ID NO 1)

| Group | Control | GXH | Pooled SEM¹ |
|---|---|---|---|
| Acetate | 51.31^{a} | 55.74^{a} | 4.19 |
| Propionate | 9.21^{a} | 9.23^{a} | 0.91 |
| I sobutyrate | 0.66^{a} | 0.55^{a} | 0.09 |
| Butyrate | 10.92^{b} | 14.49^{a} | 1.74 |
| I sovalerate | 1.28^{a} | 1.38^{a} | 0.17 |
| Valerate | 1.22^{a} | 1.32^{a} | 0.13 |
| Total SCFA | 75.99^{a} | 82.72^{a} | 6.17 |

¹SEM = Standard Error Mean.
^{ab}Mean values within a column not sharing a common letter differ significantly (P < 0.05; Tukey-Kramer HSD) (n = 24).

Table 6 shows a greater than 8% increase in acetate content, a greater than 30% increase in butyrate content, and a total increase in SOFA content of approximately 9%.

In Vitro Fermentations with Cecal Content for SCFA Analysis

The effect of the GH30 on SOFA production was tested by in vitro fermentation with inoculum from pooled cecal content from two 29 d-old broilers (table 7). Addition of the enzyme at 10 mg EP/kg was associated with a significantly increased (P<0.05) butyrate concentration (+4.1 mM) after 48 h of anaerobic incubation while propionate and valerate were significantly lowered (P<0.01). No measurable lactate was present in the samples (data not shown).

Conclusion

The GAX-specific glucuronoxylan hydrolase improved performance and morphological gut health parameters in broilers fed a maize/DDGS/soy diet. NSP, cecal SOFA and in vitro fermentation data suggest high solubilisation of prebiotic AXOS by the enzyme that can be fermented by the resident microbiota.

The invention is further defined by the following numbered paragraphs:
1. A method of improving the feed conversion ratio of an animal feed comprising maize comprising adding GH30 glucuronoxylan hydrolase to said animal feed.
2. A method of improving the feed conversion ratio of a maize-based animal feed, comprising the adding GH30 glucuronoxylan hydrolase to said maize-based animal feed
3. The method according to paragraph 1, wherein the maize is fibrous maize.
4. The method according to any one of paragraphs 1 to 3 wherein the feed further comprises maize DDGS.
5. The method according to any one of paragraphs 1 to 4, wherein the feed further comprises soybean meal.
6. The method according to any one of paragraphs 1 to 5, wherein the feed comprises maize in an amount of 100 to 1000 g/kg feed, such as 100 to 800 g/kg feed, such as 200 to 800 g/kg feed, such as 200 to 600 g/kg feed, such as 300 to 600 g/kg feed.
7. The method according to any one of paragraphs 1 to 5 wherein the animal is a monogastric animal.
8. The method according to any one of the preceding paragraphs wherein the animal is selected from the group consisting of poultry and swine.
9. The method according to any one of the preceding paragraphs wherein the animal is chicken.
10. The method according to any one of the preceding paragraphs wherein the animal feed comprises 2 to 100 ppm of the GH30 glucuronoxylan hydrolase per kg of feed, such as 2 to 50 ppm, such as 2 to 40 ppm, such as 2.5 to 25 ppm, such as 5 to 20 ppm.
11. A method of improving the feed conversion ratio of monogastric animals comprising the use of GH30 glucuronoxylan hydrolase in a maize-based animal feed.
12. A method of generating a prebiotic in-situ in a maize-based animal feed comprising the use of a GH30 glucuronoxylan hydrolase added to said feed.
13. The method of generating a prebiotic in-situ according to paragraph 11 wherein the prebiotic is an arabinoxylan oligosaccharide and polysaccharides.
14. A method of decreasing the insoluble maize fraction in a maize-based animal feed comprising the addition of a GH30 glucuronoxylan hydrolase.
15. A method of improving the intestinal health of a monogastric animal comprising the administration of an enzyme-enriched maize-based animal feed to said animal wherein said animal feed comprises the enzyme GH30 glucuronoxylan hydrolase.
16. The method according to paragraph 14 wherein the GH30 glucuronoxylan hydrolase degrades the non-starch polysaccharides of said maize so as to generate prebiotic oligomers and polymers, prebiotic oligomers and polymers comprising arabinoxylan oligosaccharides.
17. A method of improving the intestinal health of a monogastric animal by in-situ production of arabinoxylan oligosaccharides and polysaccharides.
18. A method for the in-situ production of prebiotics in monogastric animals comprising the administration of an enzyme-enriched maize-based animal feed to said animal wherein said animal feed comprises the enzyme GH30 glucuronoxylan hydrolase.
19. A method for improving intestinal health in a monogastric animal, said method comprising increasing the levels of cecal butyrate levels in situ in said animal said method comprising the administration of an enzyme-enriched maize-based animal feed to said animal wherein said animal feed comprises the enzyme GH30 glucuronoxylan hydrolase.
20. A method for improving intestinal health in a monogastric animal said method comprising altering the microbiota composition in said animal by administration of an enzyme-enriched maize-based animal feed to said animal wherein said animal feed comprises the enzyme GH30 glucuronoxylan hydrolase.
21. The method according to paragraph 19 wherein the microbiota composition of said animal is altered in that, at least, Baceteroide levels are reduced and Lachnospiraceae and/or Ruminococcaceae levels are increased.
22. A method of feeding an animal, comprising the administration of an enzyme-enriched maize-based animal feed to said animal wherein said animal feed comprises the enzyme GH30 glucuronoxylan hydrolase.
23. A method of causing a butyrogenic effect in a monogastric animal comprising the administration of an enzyme-enriched maize-based animal feed to said animal wherein said animal feed comprises the enzyme GH30 glucuronoxylan hydrolase.
24. A method of preparing an animal feed comprising maize comprising adding GH30 glucuronoxylan hydrolase to said animal feed.
25. A method of improving feed efficiency of maize-based animal feed, comprising adding GH30 glucuronoxylan hydrolase to said animal feed.
26. A method of improving nutrient digestibility of in a monogastric animal, comprising administration of an enzyme-enriched maize-based animal feed to said animal wherein said animal feed comprises the enzyme GH30 glucuronoxylan hydrolase.
27. The method according to any of paragraphs 1 to 25 wherein the GH30 glucuronoxylan hydrolase originates from Bacillus subtilis.
28. An enzyme-enriched animal feed comprising GH30 glucuronoxylan hydrolase and maize wherein the feed comprises maize in an amount of 100 to 1000 g/kg feed and GH30 glucuronoxylan hydrolase in an amount of 2 to 100 ppm per kg of feed.
29. Use of GH30 glucuronoxylan hydrolase to prepare an enzyme-enriched animal feed, wherein said animal feed is a maize-based animal feed.
30. The enzyme-enriched animal feed according to paragraph 27, the use according to paragraph 28 or the method according to any of paragraphs 1 to 26, wherein the GH30 glucuronoxylan hydrolase (EC 3.2.1.136) is a GH30_8 glucuronoxylan hydrolase.
31. The enzyme-enriched animal feed according to paragraph 27, the use according to paragraph 28 or the method according to any of paragraphs 1 to 26, wherein the GH30 glucuronoxylan hydrolase is a polypeptide have xylanase activity selected from
   a. a polypeptide having at least 80% sequence identity, such as at least 85% sequence identity, such as at least 90% sequence identity, such as at least 95% sequence identity, such as at least 98% sequence identity, such as at least 99% sequence identity to SEQ ID NO 1;
   b. a polypeptide having at least 80% sequence identity, such as at least 85% sequence identity, such as at least 90% sequence identity, such as at least 95% sequence identity, such as at least 98% sequence identity, such as at least 99% sequence identity to SEQ ID NO 2; and
   c. a polypeptide having at least 80% sequence identity, such as at least 85% sequence identity, such as at least 90% sequence identity, such as at least 95% sequence identity, such as at least 98% sequence identity, such as at least 99% sequence identity to SEQ ID NO 3.
31. The enzyme-enriched animal feed according to paragraph 27, the use according to paragraph 28 or the method according to any of paragraphs 1 to 26, wherein the GH30 glucuronoxylan hydrolase comprises, consists essentially of, or consists of SEQ ID NO 1, SEQ ID NO 2; or SEQ ID NO 3.
32. The enzyme-enriched animal feed according to paragraph 27, the use according to paragraph 28 or the method according to any of paragraphs 1 to 26, wherein one or more additional enzymes are further comprised, added or administrated.
33. The enzyme-enriched animal feed, the use or the method according to paragraph 33, wherein the one or more additional enzymes are selected from the group comprising of phytase (EC 3.1.3.8 or 3.1.3.26); xylanase (EC 3.2.1.8); galactanase (EC 3.2.1.89); alpha-galactosidase (EC 3.2.1.22); protease (EC 3.4); phospholipase A1 (EC 3.1.1.32); phospholipase A2 (EC 3.1.1.4); lysophospholipase (EC 3.1.1.5); phospholipase C (3.1.4.3); phospholipase D (EC 3.1.4.4); amylase such as, for example, alpha-amylase (EC 3.2.1.1); arabinofuranosidase (EC 3.2.1.55); beta-xylosidase (EC 3.2.1.37); acetyl xylan esterase (EC 3.1.1.72); feruloyl esterase (EC 3.1.1.73); cellulase (EC 3.2.1.4); cellobiohydrolases (EC 3.2.1.91); beta-glucosidase (EC 3.2.1.21); pullulanase (EC 3.2.1.41), alpha-mannosidase (EC 3.2.1.24), mannanase (EC 3.2.1.25) and beta-glucanase (EC 3.2.1.4 or EC 3.2.1.6), or any mixture thereof.
34. The enzyme-enriched animal feed according to paragraph 27, the use according to paragraph 28 or the method according to any of paragraphs 1 to 26, wherein one or more additional enzymes are further comprised, added or administrated. one or more microbes, one or more vitamins, one or more minerals, and/or one or more amino acids.
35. A method for improving intestinal health in a monogastric animal comprising feeding said animal a maize-based feed, wherein said maize-based feed is enriched with GH30 glucuronoxylan hydrolase.

1. A method of improving the feed conversion ratio of an animal feed comprising maize and adding GH30 glucuronoxylan hydrolase to said animal feed.

2. The method according to claim 1, wherein the maize is fibrous maize.

3. The method according to claim 1 wherein the feed further comprises maize DDGS.

4. The method according to claim 1 wherein the animal is a monogastric animal, preferably poultry and swine.

5. A method of generating a prebiotic in-situ in a maize-based animal feed comprising the use of a GH30 glucuronoxylan hydrolase added to said feed.

6-12. (canceled)

13. The method according to claim 1, wherein the GH30 glucuronoxylan hydrolase originates from Bacillus subtilis.

14. An enzyme-enriched animal feed comprising GH30 glucuronoxylan hydrolase and maize wherein the feed comprises maize in an amount of 100 to 1000 g/kg feed and GH30 glucuronoxylan hydrolase in an amount of 2 to 100 ppm per kg of feed.

15. (canceled)

16. The enzyme-enriched animal feed according to claim 14, wherein the GH30 glucuronoxylan hydrolase (EC 3.2.1.136) is a GH30_8 glucuronoxylan hydrolase.

17. The enzyme-enriched animal feed according to claim 14, wherein the GH30 glucuronoxylan hydrolase is a polypeptide have xylanase activity selected from
a. a polypeptide having at least 80% sequence identity to SEQ ID NO 1;
b. a polypeptide having at least 80% sequence identity to SEQ ID NO 2; and
c. a polypeptide having at least 80% sequence identity to SEQ ID NO 3.


SEQUENCE LISTING (21 sequences)

SEQ ID NO: 1 - protein, 391 aa, Bacillus subtilis
  PEPTIDE (1)..(391)
AASDVTVNVSAEKQVIRGFGGMNHPAWAGDLTAAQRETAFGNGQNQLGFSILRIHVDENRNNWYKEVETAKSAVKHGAIVFASPWNPPSDMVETFNRNGDTSAKRLKYNKYAAYAQHLNDFVTFMKNNGVNLYAISVQNEPDYAHEWTWWTPQEILRFMRENAGSINARVIAPESFQYLKNLSDPILNDPQALANMDILGTHLYGTQVSQFPYPLFKQKGAGKDLWMTEVYYPNSDTNSADRWPEALDVSQHIHNAMVEGDFQAYVWWYIRRSYGPMKEDGTISKRGYNMAHFSKFVRPGYVRIDATKNPNANVYVSAYKGDNKVVIVAINKSNTGVNQNFVLQNGSASNVSRWITSSSSNLQPGTNLTVSGNHFWAHLPAQSVTTFVVNR

SEQ ID NO: 2 - protein, 391 aa, Bacillus subtilis
AANDVTVNISAEKQVIRGFGGMNHPAWVGDLTAAQRETAFGNGQNQLGFSILRIHVDENRNNWYKEVETAKSAIKHGAIVFASPWNPPSNMVETFNHNGDTSAKRLRYDKYAAYAQHLNDFVTFMKSNGVNLYAISIQNEPDYAHEWTWWTPQEILRFMRENAGSINARVIAPESFQYLKNLSDPILNDPQALANMDILGTHLYGTQVSQFPYPLFKQKGAGKDLWMTEVYYPNSDNNSADRWPEALDVSQHIHNSMVEGDFQAYVWWYIRRSYGPMKEDGTISKRGYNMAHFSKFVRPGYVRIDATKNPNPNVYVSAYKGDNKVVIVAINKSNTGVNQNFVLQNGSASQVSRWITSSNSNLQPGTNLKVTDNHFWAHLPAQSVTTFVVIR

SEQ ID NO: 3 - protein, 392 aa, Bacillus subtilis
  PEPTIDE (1)..(391)
AASDATVRLSAEKQVIRGFGGMNHPAWIGDLTAAQRETAFGNGQNQLGFSILRIHVDENRNNWYREVETAKSAIKHGAIVFASPWNPPSDMVETFNRNGDTSAKRLRYDKYAAYAKHLNDFVTFMKNNGVNLYAISVQNEPDYAHDWTWWTPQEILRFMKENAGSINARVIAPESFQYLKNISDPIVNDPKALANMDILGAHLYGTQLNNFAYPLFKQKGAGKDLWMTEVYYPNSDNHSADRWPEALDVSHHIHNSMVEGDFQAYVWWYIRRSYGPMKEDGTISKRGYNMAHFSKFVRPGYVRVDATKSPASNVYVSAYKGDNKVVIVAINKNNSGVNQNFVLQNGSVSQVSRWITSSSSNLQPGTNLNVTDNHFWAHLPAQSVTTFVANLR

SEQ ID NO: 4 - protein, 185 aa, Bacillus subtilis
  PEPTIDE (1)..(185)
ANTDYWQNWTDGGGTVNAVNGSGGNYSVNWSNTGNFVVGKGWTTGSPFRTINYNAGVWAPNGNAYLTLYGWTRSPLIEYYVVDSWGTYRPTGTYKGTVYSDGGTYDVYTTTRYDAPSIDGDKTTFTQYWSVRQSKRPTGSNATITFSNHVNAWKRYGMNLGSNWSYQVLATEGYRSSGSSNVTVW

SEQ ID NO: 5 - protein, 185 aa, Bacillus subtilis
  PEPTIDE (1)..(185)
ASTDYWQNWTDGGGIVNAVNGSGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDRTTFTQYWSVRQSKRPTGSNATITFSNHVNAWKSHGMNLGSNWAYQVMATEGYQSSGSSNVTVW

SEQ ID NO: 6 - protein, 557 aa, Clostridium acetobutylicum
  mat_peptide (1)..(557)
ASNVMVNLASKKQVIRGFGGMNSVAWAGDLTAAQRETAFGNGNNQLGLSVVRIFVDDNKNNWYKELPTAKSAIAHGSIVFATPWNPPSSMTETFNRNGEKAKRLRYDKYGDYAKYLNDFVSYMKNNGVNLYAISVQNEPDYGRDWTWWTPQEVLRFMRDYAGSINCRVMSPESFSYQKNMYDPILNDPKALANMDILGTHTYGTQVKDFPYPLFKQKAAGKDLWMTEVYVPNSDANSADRWPEALEVANHINNAMVEGDFQAYVWWYIRRSYGLIKENGAISKRGYMMAHFSKFVRPGYVRVDATKNPVGNVYVSAYTGNNKVVIVAINKGTYPVNQSFNIQNSTVSNVSSWVTSGTLNMAKTNSNINAANGRFNASLPAQSVTTFVADLNSTKPTTNPTTNPTPGSTVTLNNGWYYIKNINAQKYLQVANNTGKAGQNVELGSGSGVAGQKWYLTNTGDGYITLKNALGNYMLDVSYGENKDGSNIQIFNAYSGDSQKFAVKASSKNGQYSVATKSSNGSKVLDDYNFGTADGTNVCQWTYGGNANQLWVFEPTNN

SEQ ID NO: 7 - protein, 382 aa, Pseudoalteromonas tetraodonis
  mat_peptide (1)..(382)
SNVTINFNTQYQQIDGFGGMNAPGWINDLTPAQATKAFGTGNGEMGLSIMRMRIAPDSNQWYKQVPTAKIAKSYGAKLLASPWSPPAYMKSNNNLNNGGKLEKTHYWGYTNHLMDFTNYMASQGASVYALSLQNEPDWHPEYESCDWSASDFVNYLNDQGWRLDPALKILAPESLGFNKALSDPILNNSVANNYVDIIGGHLYGVSPSNYPLALQKGKKLWMTEHYTDNEDGNNWNASIDVGLELHQSMVSNYSAYIWWYIRRSYGLMSEDGNVSKRGYIMAQFSKYIRPGYTRIGATEMPENNVYVTAYKNNAGKLVIVVVNKSGSPKALDFTLQNGTVNTLTKYSTSASMNMEYRGKSTVSNNRFSAYADAWAVQTFVSN

SEQ ID NO: 8 - protein, 391 aa, Paenibacillus sp-19179
  mat_peptide (1)..(391)
ASDAIVNISAEKQVIRGFGGINHPVWIGDLTAAQRETAFGNGNNQLGFSILRIYVHEDRNQWYREVETAKRAIALGAIVFASPWNPPADMVETFNRNGDPSAKRLRYDKYAAYAQHLNDFVTYMRNNGVNLYAISVQNEPDYAHDWTWWTPQEMLRFMKENAGSINTRVIAPESFQYLKNMSDPILNDPQALANMDILGAHLYGTQVSNFAYPLFKQKGAGKDLWMTEVYYPNSDNNSADRWPEALDVSYHIHNAMVEGDFQAYVWWYIRRSYGPMKEDGTISKRGYAMAHFSKFVRPGYVRVEATKNPETNVYVSAYKGNKKLVIVAVNKNNSGVNQNFVLPNASVSKISRWITSGSSNLQPGTELTMTGGNFWAHLPAQSVTTFVADLG

SEQ ID NO: 9 - protein, 383 aa, Pectobacterium carotovorum subsp. Carotovorum
  mat_peptide (1)..(383)
DTVKIDAKTSYQIIQGFGGMNAPGWINDLTTEQVNTAFGNDTGQIGLSIMRMRIDPDANRWNIQVSSARQASLLGAKLMATPWTPPAYMKSNKSLINGGRLLSEHYSGYTEHLLKFSNFMQTNNAPLYAISIQNEPDWKPDYESCEWNGNDFKNYLKSQGSKFGSLKVIVGESLNFNHSLTDPTLNDSEAAKHVAIVGGHLYGTTPKPYPLAQNKGKEVWMTEHLVDSKQSANNWSSALEVASEMNASMVANYNAYVWWYIRRSYGLLTEDGKVSKRGYVMAQYAKFVRPGFQRIQATENPQANVHLTAYKNSEGKMVIVAINTNDSDQLLSLNISNHTVSKFEKYSTSAILNVEYGGTYKVDSNGKSSVWLNPLSVTTFVGK

SEQ ID NO: 10 - protein, 565 aa, Ruminococcus sp. CAG330
  mat_peptide (1)..(565)
ADVCVIDTDTEHQMIRGFGGINHPEWAGDLTQAQRQTAFGNGENELGLTVLRVFVNPDSSQWSRALPTAQFATQMGVTVFASPWEPPANLTESGGSNGKLHLPKSNYAAYAKHLNDFGTYMKNNNVDLYAISVQNEPDYASEWTYWSTDETTDFIANYGDQITSTRLMSPESFQYAPENASWVSDGGKKFYRKILNNSKAMANCDVFGTHFYGTQRSWMDFPDLENSGKEIWMTEVYVPNSDKDSANRYPEALQVSENIHNAMVVGNMSAYTWWYIRRNYGLMTEDGKISKRGYCMAQYSKYVRPGDVRIDATEQPADNVYVSAYKGDDNQVTIVAINKGTESYSQQFAVDADAQITEVDRYRTSASENLAKTENMEHDSSSFWAQLPAESVSTFVVTLEDQPVEPDENGYYFHDTFESDNCDWQGHGSADITLSGRIPYQGTNALLVQNRASAWNGAEKVLPAKAFQAGKEYSFSVCLNYMDGESSKNAALSLQYTDAAGETKYARIASASAAKGNYVQLANPSFKLPDGGKNFKIYVETEGDTDNFYIDEAIGAVKGTAIEGP

SEQ ID NO: 11 - protein, 396 aa, Streptomyces sp-62627
  Streptomyces sp-62627 (1)..(396)
SRTPSAAATSVTVDPSATRQTIRGFGGMNHPLWIGDLTPAQRDTAFGNGEGQLGFSVLRIPVSEDRANWSREVATAKRATELGAIVFASPWNPPANMVETFVRGQQTDAKRLRHSMYGAYAQHLNDFVAFMKSNGVNLYAISVQNEPDYAHDWTWWTPSEMTRFLRENAGSISTKVIAPESFQYVKTFSDPILNDAAALANLDILGAHLYGTSFQNFPYPLFKQKGGGKELWMTEVYHPNSSDSADLWPQALDVAEHIHRAMVDAEFQAYVWWYIRRGYGPMREDGRISKRGAGMAHFSKFVRPGHVRVAVTPAPQPNVYLSAYKGGGSRVVVVAVNKGASPVSQQFTLNNNNSSGVSSWVTDASRNLASQGRITVANGAFTARLPAQSVTTLVTG

SEQ ID NO: 12 - protein, 392 aa, Clostridium saccharoperbutylacetonicum
  mat_peptide (1)..(392)
ASNATINLSAQKQVIRGFGGINLPAWAGDLTAAQRETAFGNGDNQLGLSVLRIYVDDNKNNWYKELATAKKAIEHGAIVFATPWNPPAYMTEKFNRNGDTNAKRLRYDKYAAYAQHLNDFVSYMKNNGVNLYAISVQNEPDYGKEWTWWTPQEILRFIKENAGSINCRVMSPESFSYQKNMYDPILNNPQALANMDILGTHTYGTRVNDFAYPLFKQKGAGKELWMTEVYVPNSDTNSADRWPEALDVADHINNAMVEGDFQAYVWWYIRRSYGFIKEDGNVSKRGYMMAHFSKFVRPGYVRVDATKNPTPNVYLSAYKGNNKVVIIAINKGTSDVKQSFTMPNSKVSSVSSWQTTATANLAKSASNTNVYNGNFTATLPAQSVTTFVGDIK

SEQ ID NO: 13 - protein, 413 aa, Paenibacillus panacisoli
  mat_peptide (1)..(413)
ASDAVINLSAQKQVIRGFGGINHPAWIGDLTAAQRETAFGNGQNQLGFSILRVYIDPDRNNWSREVATAKKAIEKGALVFASPWNPPSSMVETFNRNGDRNAKRLRYDKYAAYAQHLNDFVTYMKNNGVNLYAISVQNEPDYAHEWTWWTPQEILRFMKENAGSINCKVMAPESFQYLKNISDPILNDPQALANMDILGAHLYGTQVSNFAYPLFKQKGAGKELWMTEVYYPNSDNNSADRWPEALEVSHHMHNAMVEGDFQAYVWWYIRRSYGPMKEDGTISKRGYNMAHFSKFIRPGYVRVDATKNPDTNVHVSAYKGNNKVVIVAINRGTTAVNQNFVLQNGRAATLSRWITDANRNLAPESNLNASSGSFFAHLPAKSVTTFVGDLTGSTLNIEDAALTNSVTQDTYSK

SEQ ID NO: 14 - protein, 398 aa, Human Stool metagenome
  PEPTIDE (1)..(398)
AQAASDAVINLNNTHQEIMGFGGMNHPTWAGDLTSSQRETAFGNGTNQLGFQVLRIWVDSDRNNWYKELATAKAALAKGAIVFATPWNPPSNLCETFYKNGSANAKRLKHDKYAAYAQHLNDFVTYMRNNGVELYGISVCNEPDYGHDWTWWTESEVVTFLKYYAGSINCRIIAPESFSYQKSYYDAIINDSQALAQVDIIGTHLYGTSYNNFSYPLYHQKASSKQLWMTEVYTPNSTSSADKWPEAINVAEHIHKAMVNDFQTYVWWYIRRSYGPMKEDGTLSKRGYCMAQFSKFIRRGYKRVDATENPNNGVYVSAYTGDGKAVIVAVNSGSSDCSQSFTIKGKTLKNVDRYRTSGSENLAKTSNLELSGNGFWAYLPANSVSTFVCTVENSSSNP

SEQ ID NO: 15 - protein, 382 aa, Vibrio rhizosphaerae
  mat_peptide (1)..(382)
GSVYINFNTEYQEIDGFGAMNAPGWVNDLTSAQATKAFGNGDGQMGLSIMRMRIDPDSNQWYRQVPTAQIAYSYGAKLLATPWSPPAYMKTNNNVNNGGKLKKEHYWGYTDHLMDFTNYMAGKNAPIYALSIQNEPDWHPNYESCDWSGADFVNYLNDQGWRLDSSLKILAPESLGFNPALSDPILKDSVASSHIDIIGGHLYGVQPRNYPLALQKGKKLWMTEHYTDTDNANIWDKAMNVGLELHQSMVSNYSAYIWWYLRRSYGMLTEDGNISKRGYIMSQFSKFIRPGDVRIAATEVPESNVYVTAYKNRSGKLVIAVVNKTNSHKKLDFTLQNGTVGSMTKYVTSASQNVGYAGKYAVSNNRFTAYADPLSVQTFVSE

SEQ ID NO: 16 - protein, 391 aa, Bacillus subtilis; identical to SEQ ID NO: 1
  PEPTIDE (1)..(391)

SEQ ID NO: 17 - protein, 391 aa, Bacillus amyloliquefaciens
  PEPTIDE (1)..(391)
AANDVTVNISAERQVIRGFGGMNHPAWVGDLTAAQRETAFGNGQNQLGFSILRIHVDENRNNWYKEVETAKSAIKHGAIVFASPWNPPNDMVETFNHNGDTSAKRLRYDKYAAYAQHLNDFVNFMKSNGVNLYAISIQNEPDYAHEWTWWTPQEILRFMRENAGSINARVIAPESFQYLKNLSDPILNDPQALANMDILGTHLYGTQVSQFPYPLFKQKGAGKDLWMTEVYYPNSDNNSADRWPEALDVSQHIHSSMVEGDLQAYVWWYIRRSYGPMKEDGTISKRGYNMAHFSKFVRPGYVRIDATKNPNPNVYVSAYKGDNKVVIVAINKTNTGVNQNFVLQNGSASQVSRWITSSNSNLQPGTDLKVTDNHFWAHLPAQSVTTFVVKR

SEQ ID NO: 18 - protein, 392 aa, Bacillus licheniformis; identical to SEQ ID NO: 3
  PEPTIDE (1)..(392)

SEQ ID NO: 19 - protein, 391 aa, Bacillus subtilis; identical to SEQ ID NO: 2
  PEPTIDE (1)..(391)

SEQ ID NO: 20 - protein, 393 aa, Paenibacillus pabuli
  PEPTIDE (1)..(393)
AASDVTVNLSSEKQLIKGFGGINHPNWIGDLTPSQRDTAFGNGQNQLGFSILRIYIDDNKNNWYKEIPTAKRAIEQGAIVFASPWNPPSDMVETFNRNGDTAAKRLKYDKYAAYAQHLNDFVSYMKSNGVNLYAISVQNEPDYAHDWTWWTPQEMLRFMKDYAGSITGTKVMAPESFSYLKEMSDPILNDPQALANMDILGAHTYGTQFSNFPYPLFKQKGAGKELWMSEVYYPNSNANSADHWPEALDVSYHIHHAMVEADFQAYVWWYIRRQYGPMKEDGTISKRGYNMAHFSKFVRPGFVRVDATKNPDTQTFISAFKGDNKVVIVAINRGTSAVNQKFVLQNGNASNVSSWVTDSTRNLAAGSSIIMTGNTFTAQLPSQSVTTFVAQLN

SEQ ID NO: 21 - protein, 391 aa, Bacillus amyloliquefaciens
  PEPTIDE (1)..(391)
AASDATVNISAERQVIRGFGGMNHPAWIGDLTAPQRVTAFGNGQNQLGFSVLRIHVDENRNNWYKEVETAKSAIKHGAIVFASPWNPPNDMVETFNHNGDTSAKRLRYDKYAAYAQHLNDFVNFMKSNGVNLYAISMQNEPDYAHEWTWWTPQEILRFMRENAGSINTRVIAPESFQYLKNISDPILNDPQALRNMDILGTHLYGTQVSQFPYPLFKQKGGGKELWMTEVYYPNSDNYSADRWPEALGVSEHIHHSMVEGDFQAYVWWYIRRSYGPMKEDGMISKRGYNMAHFSKFVRPGYVRIDATKNPEPNVYVSAYKGDNKVVIVAINKNNTGVNQNFVLQNGTASQVSRWITSSSSNLQPGTDLKVTDNHFWAHLPAQSVTTFVVKR